(19) United States
(12) Patent Application Publication
(10) Pub. No.: US 2022/0090046 A1
(43) Pub. Date: Mar. 24, 2022

(54) Encapsulated Cells

(71) Applicant: The Australian National University, Acton, Australian Capital Territory (AU)

(72) Inventors: Thomas HUBER, Acton, Australian Capital Territory (AU); Damian VAN RAAD, Acton, Australian Capital Territory (AU)

(21) Appl. No.: 17/422,924

(22) PCT Filed: Jan. 24, 2020
(86) PCT No.: PCT/AU2020/050050

§ 371 Date: Jul. 14, 2021

(30) Foreign Application Priority Data
Jan. 25, 2019 (AU) 2019900240

(51) Int. Cl. C12N 11/04; C12N 11/10; C12N 9/36

(52) U.S. Cl. CPC C12N 11/04; C12Y 302/01017; C12N 9/2462; C12N 11/10

(57) ABSTRACT

Provided herein are encapsulated cells that comprise a microbial cell modified to express a cytolytic enzyme and encapsulated with one or more layers of a cationic polymer and one or more layers of an anionic polymer that may be used in cell-free protein synthesis. To this end, methods of utilizing such encapsulated cells in synthesizing a target protein, degrading a contaminant in a contaminated environment and detecting an analyte in a sample are also disclosed herein. Further provided herein, are methods of producing such encapsulated cells.

### FIELD

THIS INVENTION relates to encapsulated cells and methods of making same. More particularly, this invention relates to encapsulated cells for use in cell free protein synthesis systems.

### BACKGROUND

Recombinant protein production is generally performed by two systems; an in vivo (living) or in vitro (non-living) system. Escherichia coli (E. coli) is typically used as a model organism for large scale production of protein using both in vivo and in vitro systems [1].

In vitro protein synthesis or Cell-Free Protein Synthesis (CFPS) is the functional use of complex biological transcription/translation processes without the use of intact cells [2]. It usually includes the use of a cellular lysate that holds all the necessary macromolecules that facilitate translation, transcription, protein folding and energy production [3]. These factors can be used to direct synthesis of a specific target protein defined by the experimenter.

CFPS E. coli lysates are commonly defined as ‘S30 extracts’ coined through the preparation process. The cells are harvested at the middle of the exponential phase of low density growth and then processed using a French press at 12,000 psi. The lysate is then centrifuged at 30,000 g twice to remove cell wall fragments, genomic DNA, mRNA and then the supernatant is collected, hence the term ‘Supernatant 30,000 g’ or S30 [4]. There are issues surrounding lysate preparation as cell extracts have issues with consistent activity between batches [5]. Accordingly, there exists a need for improved methods of CFPS.

### SUMMARY

The present inventors have discovered a method of producing encapsulated cells by encapsulating a microbial cell that has been modified to express a cytolytic enzyme for degradation of a cell wall thereof and effectively autolysis of the microbial cell. This method represents an efficient and cost effective means of producing non-living encapsulated cells that can be used, for example, in systems of cell-free protein synthesis (CFPS).

In a first aspect, the invention provides a method of producing encapsulated cells, including the steps of:

(i) providing a plurality of microbial cells, the microbial cells modified to express a cytolytic enzyme;

(ii) contacting the microbial cells with a cationic polymer;

(iii) contacting the microbial cells with an anionic polymer;

to thereby produce encapsulated cells.

Suitably, the method of the present aspect further includes the step of facilitating at least partial hydrolysis of a cell wall of the microbial cells by the cytolytic enzyme. In some embodiments, the step of facilitating hydrolysis of the cell wall comprises subjecting the encapsulated cells to a freeze-thaw cycle. In particular embodiments, the step of facilitating hydrolysis of the cell wall comprises lyophilisation of the encapsulated cells.

In a second aspect, the invention provides an encapsulated cell produced by the method of the first aspect.

In a third aspect, the invention provides an encapsulated cell comprising a microbial cell modified to express a cytolytic enzyme, the microbial cell encapsulated with one or more layers of a cationic polymer and one or more layers of an anionic polymer.

Suitably, a cell wall of the microbial cell has been at least partly hydrolysed by the cytolytic enzyme.

In one embodiment, the encapsulated cell has been subjected to a freeze-thaw cycle.

In certain embodiments, the encapsulated cell is lyophilised.

In particular embodiments, the encapsulated cell of the present aspect is for use in synthesizing a target protein.

Referring to the first and third aspects, the cationic polymer suitably is or comprises chitosan.

In a particular embodiment of the first and third aspects, the anionic polymer is or comprises alginate.

For the aforementioned aspects, the microbial cells suitably are or comprise E. coli.

In one preferred embodiment of the first and third aspects, the cytolytic enzyme is or comprises an endolysin.

In a fourth aspect, the invention provides a method of synthesizing a target protein, including the steps of:

providing one or a plurality of encapsulated cells, the encapsulated cells comprising a microbial cell modified to express a cytolytic enzyme and encapsulated with one or more layers of a cationic polymer and one or more layers of an anionic polymer, wherein a cell wall of the microbial cell has been at least partly hydrolysed by the cytolytic enzyme and the encapsulated cells are further modified to comprise a nucleic acid sequence that encodes the target protein; and

expressing (e.g., transcribing and translating) the nucleic acid sequence, to thereby synthesize the target protein.

In a fifth aspect, the invention provides a method of degrading a contaminant in a contaminated environment, including the step of:

contacting the contaminated environment with one or plurality of encapsulated cells, the encapsulated cells comprising a microbial cell modified to express a cytolytic enzyme and encapsulated with one or more layers of a cationic polymer and one or more layers of an anionic polymer, wherein a cell wall of the microbial cell has been at least partly hydrolysed by the cytolytic enzyme, the encapsulated cells further modified to comprise a nucleic acid sequence that encodes an enzyme that is configured to at least partly degrade the contaminant;

to thereby degrade the contaminant in the contaminated environment.

In a sixth aspect, the invention provides a method for detecting an analyte in a sample, including the step of:

contacting the sample with one or a plurality of encapsulated cells, the encapsulated cells comprising a microbial cell modified to express a cytolytic enzyme and encapsulated with one or more layers of a cationic polymer and one or more layers of an anionic polymer, wherein a cell wall of the microbial cell has been at least partly hydrolysed by the cytolytic enzyme, the encapsulated cells further modified to comprise a nucleic acid sequence that encodes a marker protein, wherein the nucleic acid sequence is configured to express the marker protein upon direct and/or indirect binding with the analyte;

to thereby detect said analyte

Referring to the methods of the fourth, fifth and sixth aspects, the one or plurality of encapsulated cells is suitably that of the second and third aspects.

In a seventh aspect, the invention provides a kit for use in the method of the fourth, fifth and sixth aspects, the kit comprising the encapsulated cells of the second and third aspects and optionally instructions for use.

As used herein, the indefinite articles ‘a’ and ‘an’ are used here to refer to or encompass singular or plural elements or features and should not be taken as meaning or defining “one” or a “single” element or feature.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

By “consisting essentially of” in the context of an amino acid sequence is meant the recited amino acid sequence together with an additional one, two or three amino acids at the N- or C-terminus.

### BRIEF DESCRIPTION OF THE DRAWINGS

FIG. 1. Two ‘worm like’ polyelectrolytes expanding due to electrostatic repulsion. Adapted from [74]

FIG. 2. LbL assembly using a polyanion and a polycation to create a bilayer of polymer. Adapted from [48].

FIG. 3. Chitin (above) and its completely deacylated form chitosan (below). Adapted from [75].

FIG. 4. Alginate polymer with its two monomer units defined, α-Lguluronic acid and β-D-mannuronic acid. Adapted from [52].

FIG. 5. A ‘holin-mediated permeabilising event’ and the subsequent digestion of the peptidoglycan layer. Adapted from [62].

FIG. 6. Induced, encapsulated and lysed Xjb(DE3)* with WT nGFP722 in PBS-E buffer. The relative diffusion of GFP from an encapsulated system over the course of 24 hrs, with the half-point of GFP diffusion at 4 hours. For the final three points, error bars are too small to be seen on the graph.

FIG. 7. (A) in vitro protein production of WT 722 nGFP in encapsulated and lysed E-Cells using HMP/Maltodextrin CF buffer. We see a substantial production of GFP exceeding that of the background control of PBS-E IPTG 1 mM indicating that in-vitro protein synthesis occurred. (B) is the use of GMML (glycerol-minimal media)+1 mM of IPTG to indicate if in-vivo activity remains in E-cells, as live cells are able to produce protein in GMML. A calibration curve of pure GFP allows for estimation of GFP content in each of the graphs (provided in the supplementary information). PI inhibitor was used for all subsequent experiments.

FIG. 8. Traditional substrate level ATP generation, CK/CP in an encapsulated CFPS synthesis setting. There is a clear distinction between the control and CP/CK translation indicating that there was successful induction of protein synthesis. Encapsulated CFPS and the use of a eukaryotic generation system highlights the versatility of E-cells and its in-vitro translation.

FIG. 9. The first attempt at incorporating p-acetyl-L-phenylalanine into an encapsulated system with fluorescence points measured at three 3 hour intervals. There is clear distinction between + and − NCAA, indicating successful incorporation of PaF into amber pCDF nGFP. There is no appreciable fluorescence in the control system which corresponds well to the reported literature that fidelity was greater than 99%.

FIG. 10. Time graph of amber nGFP protein synthesis using PylRS system in encapsulated CFPS. The + NCAA time course indicated that there was amber suppression and protein synthesis, especially in comparison with the control. There seemed to some nAA suppression in the control, which was expected as the tRNA has a mutation in the acceptor stem reducing suppression affinity.

FIG. 11. SDS-PAGE gel of PPiB after encapsulated CFPS. L is protein ladder, S is supernatant, W is wash and E is eluent. 30 μl of 3 ml of eluent was used, before concentrating protein. This is the reason for the small size of the PPiB band. The relative purity of the sample should be noted, as His GraviTrap TALON columns were used and have issues with nonspecific binding.

FIG. 12. Principle of FACS: a laser shining onto a sample causing forward scatter (FSC), side scatter (SSC) and fluorescence. Positive samples that have all of the correct experimentally defined characteristics are sorted while negatives are discarded. Adapted from [76].

FIG. 13. Forward scatter or FSC, is light that refracts at low angles when it passes through a cell and can be roughly estimated as cell diameter. Adapted from [76].

FIG. 14. Side or 90° scatter (SSC) is the light that refracts at approximately 90° angle to the laser and is a measure of cellular complexity. Adapted from [76].

FIG. 15. After the first round of encapsulated CFPS the encapsulated cells were sorted to create a pool of functional aaRS. (A) shows the SSC and FSC gating we utilised to sort single microcapsules. (B) has ‘medium’ and ‘high’ gating we used to remove non-fluorescent particles i.e. unfunctional aaRS. (C) is a similar gating method to (B), although uses side scatter instead of forward scatter. (D) & (E) are a different representation of populations that exhibit fluorescence. Only 2% of particles actually exhibit fluorescence and correspond to functional synthetases.

FIG. 16. Schematic of plate based negative selection. Plasmids were extracted with light sonication and transformed with extremely competent DH10B cells, then iterative rounds of gel purification to remove contaminant plasmids such as pCDF GFP and then retransformation to recover the library plasmids.

FIG. 17. Schematic of plate based negative selection. Plasmids were extracted with light sonication and transformed with extremely competent DH10B cells, then iterative rounds of gel purification to remove contaminant plasmids such as pCDF GFP and then retransformation to recover the library plasmids.

FIG. 18. (A) Both populations of E-cell candidates (1-8) at the start of encapsulated CFPS emitting the similar fluorescent values. (B) is 20 hrs later and has a distinct difference between the two populations.

FIG. 19. Lyophilised WT 722 nGFP E-cells that have been subjected to freeze-drying.

### DETAILED DESCRIPTION

The present invention is at least partly predicated on the discovery that Escherichia coli that have been genetically modified or engineered to express a cytolytic enzyme, such as a bacteriophage-derived endolysin, can be successfully used to produce non-living encapsulated cells, which represent an efficient, cost effective and readily scalable means of performing nano-scale CFPS. As such, the present invention advantageously provides for the relatively quick and inexpensive production of encapsulated cells. Such encapsulated cells can then be utilised in an in-vitro gene and protein expression system that obviates the need for the maintenance of live microorganisms to produce biomolecules.

In one aspect, the invention therefore relates to a method of producing encapsulated cells, including the steps of:

(i) providing a plurality of microbial cells, the microbial cells modified to express a cytolytic enzyme;

(ii) contacting the microbial cells with a cationic polymer;

(iii) contacting the microbial cells with an anionic polymer;

to thereby produce encapsulated cells.

The terms “microbial cells”, “microbes” and “microorganisms” as used interchangeably herein should be understood to broadly include any microbe possessing a cell wall, inclusive of bacteria, algae (e.g., cyanobacteria) and fungi (e.g., yeasts, moulds). Regardless of the type of microbe of interest, a microbial cell may be selected that has a cell wall or membrane capable of being digested or hydrolysed by the cytolytic enzyme hereinafter described.

With respect to the present invention, the microbial cells may be considered to be isolated. For the purposes of this invention, the term “isolated” refers to material, such as the microbial cells described herein, that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form. Isolated material may also, or alternatively, be in enriched, partially purified or purified form.

It will be appreciated that the term “modified” as used herein indicates any modification of the microbial cells that facilitates their ability to express or produce a cytolytic enzyme. In specific embodiments, the microbial cells are “genetically modified” which denotes any modification of their DNA sequence or genome that facilitates the expression of the cytolytic enzyme including the modification or addition of sequences that regulate the expression of genes coding for such enzymatic activities. It is also possible to construct genetically modified microbial cells according to the invention by conventional recombinant DNA-technology including insertion of sequences coding for the cytolytic enzyme, e.g. by a chromosomally inserted gene that encodes the cytolytic enzyme together with an inducible promoter. The source of such genes that encode the cytolytic enzyme may be, for example, bacterial, fungal (e.g., yeast), viral (e.g., bacteriophage) or mammalian. In particular embodiments, the gene encoding the cytolytic enzyme is derived, at least in part, from a bacteriophage.

In some embodiments, the method of the present aspect further includes the step of modifying the plurality of microbial cells to express the cytolytic enzyme. The skilled artisan will appreciate that this may be performed by any means known in the art, such as those hereinafter described.

As generally used herein, the term “cytolytic enzyme” refers to an enzyme that is capable of initiating, causing or modulating cytolysis or cell killing. The cytolysis process caused or modulated by the cytolytic enzyme encompasses a host of biological effects, including, for example, such as rupture and dissolution or destruction of cell walls and/or membranes. In particular embodiments, the cytolytic enzyme is a hydrolytic enzyme. By “hydrolytic enzyme” is meant enzymes that are broadly catabolic enzymes that break the chemical bond between atoms of large molecule in the presence of water. Non-limiting examples of cytolytic enzymes include endolysin (lysozyme), perforin, holin, granzyme A, granzyme B, autolysins and cytolysins.

In one particular embodiment, the cytolytic enzyme is or comprises an endolysin. The term “endolysin” as used herein refers to an enzyme which is capable of hydrolysing microbial cell walls or membranes, including, for example, peptidoglycan, the outer membrane of Gram-negative bacteria with lipopolysaccharide (LPS), the bacterial cell membrane, and additional layers deposited on the peptidoglycan (e.g., outer protein layers or slimes). Endolysins are naturally coded by bacteriophages and are usually produced by them at the end of their host cycle to lyse the host cell and thereby release their offspring phages. These enzymes typically comprise at least one enzymatically active domain (EAD) having at least one of the following activities: endopeptidase, N-acetyl-muramoyl-L-alanine-amidase (amidase), N-acetyl-muramidase, N-acetyl-glucosaminidase (lysozyme) or transglycosylases. In addition, the endolysins may also contain regions which are enzymatically inactive, and bind to the cell wall of the host bacteria, the so-called CBDs (cell wall binding domains).

Suitably, the cytolytic enzyme disrupts the cell wall or membrane of the microbial cell so as to sufficiently expose the microbial cell's interior, without affecting the polymeric coating or capsule applied to the microbial cell during the encapsulation steps and thus retain the microbial cells cytoplasmic structures within the encapsulated cell.

Further to the above, the method of the present aspect may further include the step of stimulating or inducing production of the cytolytic enzyme by the microbial cells. In particular embodiments, expression of the cytolytic enzyme by the microbial cells is inducible by way of a chemical agent (e.g., IPTG, arabinose, a metal ion), inclusive of addition or depletion thereof, and/or a physical stimulus (e.g., heat shock, cold shock). Preferably, the step of inducing production of the cytolytic enzyme is performed prior to contacting the microbial cells with the cationic and/or anionic polymers.

The term “cationic polymer” refers to those polymers that have a net positive charge, such as at a particular pH, including in this definition those cationic polymers on which changes have been made such as chemical or enzymatic fragmentation, derivatisation or modification. Non-limiting examples of suitable cationic polymers are polysaccharides, proteins and synthetic polymers. Cationic polysaccharides include cationic cellulose derivatives, cationic guar gum derivatives, chitosan and derivatives thereof and cationic starches. Suitable cationic polysaccharides include cationically modified cellulose, particularly cationic hydroxyethylcellulose and cationic hydroxypropylcellulose. In one preferred embodiment, the cationic polymer is or comprises chitosan. It will be apparent to the skilled person that chitosan is a (random) linear polymer of β-1-4-D-glucosamine and N-acetyl-D-glucosamine that is typically derived from chitin in the shells of crabs and other crustaceans.

By the term “anionic polymer” is meant any polymer having a net negative charge, including in this definition those anionic polymers on which changes have been made such as chemical or enzymatic fragmentation, derivatisation or modification. Exemplary anionic polymers include hyaluronic acid, colominic acid, polysialic, chondroitin, keratan, dextrans, heparin, carrageenan, furceleranos, alginates, agar, glucomannan, gellan gum, locust bean gum, guar gum, tragacanth gum, gum arabic, xanthan gum, karaya gum, pectins, celluloses, starches, sorbitan esters and salts or fragments thereof or derivatives thereof. In one preferred embodiment, the anionic polymer is or comprises an alginate. In this regard, it will be understood that alginate is a linear copolymer of (1-4)β-D-mannuronate and alpha-L-guluronate.

Other polyions (i.e., anionic or cationic polymers) that can be utilised for performing the invention include, without limitation thereto, poly-L-lysine, carboxymethylcellulose, poly(sodium 4-styrenesulfonate), poly(allylamine hydrochloride), sodium polystyrene sulfonate, poly(styrene)-co-styrene sodium sulfonate (NaPSS), PLGA (polylactic-co-glycolic acid) and polyacrylic acid.

In particular embodiments, step (ii) of the present method (i.e., the cationic treatment step) precedes step (iii) (i.e., the anionic treatment step). To this end, it will be appreciated that the initial polymer layer of the encapsulated cells depends on the properties of the template surface. For bacteria such as E. coli, the surface of the cell is generally negatively charged due to the lipopolysaccharide (LPS) comprising the external face of the outer membrane. This makes the cell amenable to an initial coating with a cationic polymer. The order of steps (ii) and (iii), however, can be reversed (i.e. the anionic polymer is applied first) if a particular microbial cell has a positively charged surface.

It will be appreciated that steps (ii) and (iii) of the present method may proceed over one or more rounds or iterations, repeating the sequence of contacting the microbial cell with cationic and anionic polymers to achieve, for example, a desired level or thickness of polymer coating. Thus, the cationic treatment step followed by an anionic treatment step may be repeated, for example, 1, 2, 3, 4, 5, 6, 7, 8, 9 or 10 times, giving rise to an ever thicker polymer coating or capsule of the encapsulated cells.

Suitably, the resulting capsules have a pore size large enough (i.e., are sufficiently porous; e.g., up to 20 kDa, 40 kDa, 60 kDa, 80 kDa, 100 kDa, 150 kDa or any range therein) to allow macromolecules, such as an analyte, a target protein, a contaminant, a toxin, an enzyme, a marker protein, to pass through.

It will be appreciated that each of the encapsulated cells may comprise one or a plurality of microbial cells (e.g., 1, 2, 3, 4, 5, 6, 7, 8, 9, 10 etc) (or the cellular contents thereof).

In one particular embodiment, each encapsulated cell comprises a single microbial cell (or the cellular contents thereof). Such encapsulated cells advantageously provide for the ability to screen for individual encapsulated cells having homogenous genetic material in the instance that there is genetic variation between encapsulated cells within a mixture. This may not be possible if there is a plurality of microbial cells contained within each encapsulated cell, as there could be multiple encapsulated cells that contain different microbial cells and hence differing genetic material thereby restricting the ability to isolate DNA that has the same composition. Accordingly, encapsulated cells that each comprise a single microbial cell generally demonstrate similar or the same characteristics based on their substantially identical DNA composition, which can be important when such cells are utilised to produce and/or isolate a specific DNA and/or protein sequence required for a particular effect.

Referring to the above and in particular embodiments, the method of the present aspect further includes the step of facilitating at least partial hydrolysis of a cell wall of the microbial cells by the cytolytic enzyme. This may be achieved by any means or method known in the art.

By way of example, Liu et al. (Proc Natl Acad Sci USA 2009; 106:21550-4) describes an auto-inducible lysis system in E. coli using the lysis genes from Salmonella phage P22 and promoter PnrsB, which was activated by the addition of nickel. Additionally, Zhang et al. (J Microbiol Methods 2009; 79:199-204) provides an autolysis E. coli system regulated by Mg²⁺ concentration, which contained the lysis genes from λ bacteriophage and promoter Pmgt from Salmonella typhimurium. Further, Hajnal et al. (Appl Microbiol Biot 2016; 100(21):9103-10) discloses a synthetic ribosome binding site for the autolysis of E. coli and Halomonas campaniensis under environmental stresses.

Accordingly, in particular embodiments, hydrolysis of the cell wall of the microbial cells by the cytolytic enzyme is activated by way of a chemical agent (e.g., IPTG, arabinose, a metal ion), inclusive of addition or depletion thereof, and/or a physical stimulus (e.g., heat shock, cold shock).

In one preferred embodiment, the step of facilitating hydrolysis of the cell wall comprises subjecting the encapsulated cells to a freeze-thaw cycle. To this end, the step of the freeze-thaw cycle is designed or intended to create or facilitate a permeabilising or solubilising event in the cell wall or membrane of the microbial cells. The freeze-thaw cycle disrupts the cell wall or membrane of the microbial cells and creates physical holes that allow the cytolytic enzyme, such as endolysin, to access underlying layers, such as the peptidoglycan layer, and cleave bonds to cause at least partial lysis of said cell wall or membrane.

As used herein, the term “freeze-thaw cycle” refers to freezing of the encapsulated cells of the invention to a temperature below 0° C. (e.g., −70° C., −65° C., −60° C., −55° C., −50° C., −45° C., −40° C., −35° C., −30° C., −25° C., −20° C., −15° C. −10° C., −5° C., 0° C. and any range therein), maintaining the encapsulated cells in a temperature below 0° C. for a defined period of time and thawing the encapsulated cells to room temperature or body temperature or any temperature above 0° C. which enables utilisation of the encapsulated cells according to methods of the invention hereinafter described. The term “room temperature”, as used herein refers to a temperature of between 18° C. and 25° C. The term “body temperature”, as used herein, refers to a temperature of between 35.5° C. and 37.5° C., preferably 37° C. In another embodiment, the encapsulated cells that have undergone a freeze-thaw cycle are functional encapsulated cells.

According to one embodiment, freezing of the encapsulated cells is gradual. According to some embodiments, freezing of the encapsulated cells is through flash-freezing. As used herein, the term “flash-freezing” refers to rapidly freezing the encapsulated cells by subjecting them to cryogenic temperatures.

In particular embodiments, the encapsulated cells that underwent a freeze-thaw cycle were frozen for at least 30 minutes prior to thawing. According to another embodiment, the freeze-thaw cycle comprises freezing the encapsulated cells for at least 30, 60, 90, 120, 180, 210 minutes and any range therein prior to thawing. In another embodiment, the encapsulated cells that have undergone a freeze-thaw cycle were frozen for at least 1, 2, 3, 4, 5, 6, 7, 8, 9, 10, 24, 48, 72, 96, 120 hours and any range therein prior to thawing. In another embodiment, the encapsulated cells that have undergone a freeze-thaw cycle were frozen for at least 4, 5, 6, 7, 30, 60, 120, 365 days and any range therein prior to thawing.

In one particular embodiment, the step of facilitating hydrolysis of the cell wall or membrane comprises lyophilisation of the encapsulated cells. By way of example, after one or more freezing steps of a freeze-thaw cycle, the frozen encapsulated cells can then be lyophilised. The terms “lyophilisation”, “lyophilised” and “freeze drying” are well known in the art and encompass, for example, dehydration or sublimation by freezing and reducing the pressure to allow a frozen solvent in the material to sublimate directly from the solid phase to gas.

It will be appreciated that the size of the encapsulated cells will depend at least in part on the type, size, number and shape of the microbial cells and the thickness of the polymeric coating or capsule applied to the microbial cell during the encapsulation steps. In particular embodiments, the encapsulated cells have an average diameter in a range from about 0.2 μm to about 10.0 μm (e.g., about 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 μm or any range therein). More suitably, the encapsulated cells have an average diameter in a range from about 0.5 μm to about 1.5 μm.

In a related aspect, the invention provides an encapsulated cell produced by the method of the aforementioned aspect.

In another aspect, the invention provides an encapsulated cell comprising a microbial cell modified to express a cytolytic enzyme, the microbial cell encapsulated with one or more layers of a cationic polymer and one or more layers of an anionic polymer.

In one embodiment, a cell wall of the microbial cell has been at least partly hydrolysed by the cytolytic enzyme. As described above, this may be initiated at least in part by subjecting the encapsulated cell to one or more freeze-thaw cycles, so as to facilitate access to an inner portion of the cell wall, such as the peptidoglycan layer.

In certain embodiments, the encapsulated cell is lyophilised.

It will be appreciated that the cationic polymer and the anionic polymer may be any as are known in the art, such as those hereinbefore described. In one embodiment, the cationic polymer is or comprises chitosan. In another embodiment, the anionic polymer is or comprises alginate.

Again, it will be understood that the microbial cells may be any as are known in the art. In one preferred embodiment, however, the microbial cells are or comprise E. coli. As used herein, the term “Escherichia coli (E. coli)” refers to all E. coli, now known or identified in the future, inclusive of all known pathogenic and non-pathogenic strains and sub-strains.

The cytolytic enzyme may be any as are known in the art, inclusive of those hereinbefore described. In one embodiment, the cytolytic enzyme is or comprises an endolysin.

Suitably, the encapsulated cell of the present aspect is for use in synthesizing a target protein.

By “protein” is meant an amino acid polymer. The amino acids may be natural or non-natural amino acids, D- or L-amino acids as are well understood in the art.

The term “protein” includes and encompasses “peptide”, which is typically used to describe a protein having no more than fifty (50) amino acids and “polypeptide”, which is typically used to describe a protein having more than fifty (50) amino acids.

To this end, the encapsulated cell is suitably further modified to include a nucleic acid sequence that encodes the target protein.

The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as modified purines (for example inosine, methylinosine and methyladenosine) and modified pyrimidines (for example thiouridine and methylcytosine).

In a preferred form, the nucleic acid sequence encoding the target protein is in the form of a genetic construct.

Suitably, the genetic construct is in the form of, or comprises genetic components of, a plasmid, bacteriophage, a cosmid, a yeast or bacterial artificial chromosome as are well understood in the art. Genetic constructs may also be suitable for maintenance and propagation of the isolated nucleic acid in bacteria or other host cells, for manipulation by recombinant DNA technology.

For the purposes of protein expression, the genetic construct is an expression construct. Suitably, the expression construct comprises the one or more nucleic acids operably linked to one or more additional sequences in an expression vector. An “expression vector” may be either a self-replicating extra-chromosomal vector such as a plasmid, or a vector that integrates into a host genome.

By “operably linked” is meant that said additional nucleotide sequence(s) is/are positioned relative to the nucleic acid of the invention preferably to initiate, regulate or otherwise control transcription.

Regulatory nucleotide sequences will generally be appropriate for the microbial cell where expression is required. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The expression construct may also include an additional nucleotide sequence encoding a fusion partner (typically provided by the expression vector) so that the recombinant protein of the invention is expressed as a fusion protein.

In a related aspect, the invention provides a method of synthesizing a target protein, including the steps of:

providing one or a plurality of encapsulated cells comprising a microbial cell modified to express a cytolytic enzyme, the microbial cell encapsulated with one or more layers of a cationic polymer and one or more layers of an anionic polymer, wherein a cell wall of the microbial cell has been at least partly hydrolysed by the cytolytic enzyme and the encapsulated cells are further modified to comprise a nucleic acid sequence that encodes the target protein; and

expressing the nucleic acid sequence, to thereby synthesize the target protein.

Preferably, the encapsulated cell is that hereinbefore described.

In some embodiments, expression of the nucleic acid sequence by the encapsulated cells is constitutive, such as by way of a constitutive promoter. A “constitutive promoter” refers to a promoter that is capable of directing transcription of an operably linked nucleic acid sequence in the absence of a stimulus (e.g., heat shock, chemicals, light, etc.).

In other embodiments, expression of the nucleic acid is inducible, such as by way of an inducible promotor or regulatory element. An “inducible promoter” is a promoter that is capable of directly or indirectly activating transcription of one or more nucleic acid sequences or genes in response to a “regulatory agent” (e.g., doxycycline), or a “stimulus” (e.g., heat). In the absence of a “regulatory agent” or “stimulus”, the nucleic sequences or genes will not be substantially transcribed. As used herein, the terms “stimulus” and/or “regulatory agent” refers to a chemical agent, such as a metabolite, a small molecule, or a physiological stress directly imposed upon the microbial cell, such as cold, heat, toxins, or through the action of a pathogen or disease agent. A recombinant microbial cell containing an inducible promoter may be exposed to a regulatory agent or stimulus by externally applying the agent or stimulus to the cell or organism by exposure to the appropriate environmental condition or the operative pathogen. Inducible promoters initiate transcription only in the presence of a regulatory agent or stimulus. Non-limiting examples of inducible promoters include the tetracycline response element and promoters derived from the β-interferon gene, heat shock gene, metallothionein gene or any obtainable from steroid hormone-responsive genes.

In certain embodiments, the method of the present aspect further includes the initial step of modifying or genetically modifying the microbial cell to comprise the nucleic acid sequence so as to be capable of recombinantly expressing the target protein, such as by those methods hereinbefore described.

In some embodiments, the present method includes the step of isolating the target protein from the encapsulated cells. To this end, the recombinant target protein may be conveniently prepared by a person skilled in the art using standard protocols as for example described in Sambrook, et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. 1995-2009), in particular Chapters 10 and 16; and CURRENT PROTOCOLS IN PROTEIN SCIENCE Eds. Coligan et al., (John Wiley & Sons, Inc. 1995-2009), in particular Chapters 1, 5 and 6.

In one preferred form, the target protein is an enzyme capable of degrading or neutralising a toxin or contaminant. To this end, the encapsulated cells described herein may be suitable for use in bioremediation of a contaminated environment, such as soil and aqueous environments. In this regard, bioremediation refers to the conversion of toxic environment contaminating compounds into innocuous substances by way of microbial digestion. Generally, it is only necessary to add “foreign” microorganisms to a contaminated environment if the indigenous microorganisms do not possess the nucleic acid sequence/s required to create the enzymes capable of degrading the contaminant, if the contaminant is at such a high concentration as to be toxic to the natural microorganisms, or if the contaminant concentration is so low the natural level of microorganisms cannot further degrade it to an acceptable level.

It will be appreciated that the enzyme may be any as are known in the art. Nonlimiting examples of enzymes, inclusive of microbial enzymes, capable of degrading a toxin or contaminant for bioremediation purposes broadly include oxidoreductases, such as an oxygenase (e.g., monooxygenase, dioxygenase), a laccase, and a peroxidase (e.g., lignin peroxidase, manganese peroxidase, versatile peroxidase) and hydrolases, such as a lipase, a cellulase and a protease.

As the encapsulated cells of the present invention are non-living cells following hydrolysis of their cell wall by the cytolytic enzyme, they advantageously present no risk of secondary biocontamination as with foreign microorganisms, which can be a serious concern in the bioremediation of a contaminated environment when such living microorganisms are introduced therein. The enzymes of the encapsulated cells should also be more stable and demonstrate a longer shelf life when compared with isolated or purified enzymes used for the purposes of bioremediation.

In another preferred form, the target protein is a detectable marker protein. To this end, the encapsulated cells are suitably capable of detecting an analyte in a sample, such as a biological or environmental sample, upon contact therewith. The analyte may then enter one or more of the encapsulated cells and facilitate or activate expression of the marker protein. The extent of expression of the marker protein then serves as a measure of the available (“sensed”) concentration of the analyte. Genes or nucleic acid sequences coding for such marker proteins are widely used as so-called reporter genes.

In this regard, the step of expressing the nucleic acid sequence of the present method suitably includes contacting the encapsulated cells with the sample having the analyte. Further, the nucleic acid sequence encoding the marker protein is suitably operably coupled to one or more sensor or promoter sequences, specific for directly and/or indirectly binding the analyte in question. In this manner, it will be appreciated that a rapid and sensitive way to measure such expression of the marker protein is to fuse relevant promoter sequences to a reporter gene encoding the marker protein.

The marker protein (and encoding nucleic acid sequence or reporter gene) can be any as are known in the art. Exemplary marker proteins include alkaline phosphatase (phoA), chloramphenicol acetyl transferase (CAT-gene), catechol dioxygenase (xylE), bacterial luciferase, eukaryotic luciferase, beta-galactosidase, and a fluorescent protein (e.g., green fluorescent protein (GFP) yellow fluorescent protein (YFP), cyan fluorescent protein (CFP), blue fluorescent protein (BFP) and mCherry), albeit without limitation thereto.

In a related aspect, the invention provides a method of degrading a contaminant in a contaminated environment, including the step of:

contacting the contaminated environment with one or plurality of encapsulated cells, the encapsulated cells comprising a microbial cell modified to express a cytolytic enzyme and encapsulated with one or more layers of a cationic polymer and one or more layers of an anionic polymer, wherein a cell wall of the microbial cell has been at least partly hydrolysed by the cytolytic enzyme, the encapsulated cells further modified to comprise a nucleic acid sequence that encodes an enzyme that is configured to at least partly degrade the contaminant;

to thereby degrade the contaminant in the contaminated environment.

Suitably, the encapsulated cell is that hereinbefore described.

Additionally, the enzyme may be any as are known in the art, inclusive of those described previously.

In another related aspect, the invention resides in a method for detecting an analyte in a sample, including the step of:

contacting the sample with one or a plurality of encapsulated cells, the encapsulated cells comprising a microbial cell modified to express a cytolytic enzyme and encapsulated with one or more layers of a cationic polymer and one or more layers of an anionic polymer, wherein a cell wall of the microbial cell has been at least partly hydrolysed by the cytolytic enzyme, the encapsulated cells further modified to comprise a nucleic acid sequence that encodes a marker protein, wherein the nucleic acid sequence is configured to express the marker protein upon direct and/or indirect binding with the analyte;

to thereby detect said analyte.

It would be appreciated that certain embodiments of this aspect may be used for detecting and/or monitoring the levels of one or more analytes in a subject, either directly (e.g., in vivo or in situ) or indirectly, such as from a biological sample derived from the subject. Further embodiments of this aspect may be used in detecting and/or monitoring the presence of one or more analytes in an environmental sample.

For the present aspect, the term “contacting” can refer to any suitable means for delivering, or exposing, the sample to a plurality of the encapsulated cells described herein. By way of example, the encapsulated cells (e.g., suspended in a solution) may be added directly to the sample. In some embodiments, the term “contacting” can further comprise mixing the sample with the encapsulated by any means known in the art (e.g., vortexing, pipetting, and/or agitating). This can further comprise incubating the sample together with the encapsulated cells for a sufficient amount of time, for example, to allow binding of the marker protein to the target analytes. The contact time can be of any length, depending on the binding affinities and/or concentrations of the marker proteins and/or the analytes, and/or incubation conditions (e.g., temperature).

Further to the above, the present method may further include the step of detecting the marker protein.

Suitably, the encapsulated cell is that hereinbefore described.

In yet a further aspect, the invention resides in a kit for use in one or more of the methods of the aforementioned aspects, the kit comprising the encapsulated cells of the second mentioned aspect and optionally instructions for use.

Referring to the aforementioned aspects, it will be appreciated by the skilled artisan that general any kind of cell (in the context of its biological definition), in addition to microbial cells, can be used in relation to the invention described herein. Therefore, as generally used herein, the term “cell” refers to microbial cells, such as those hereinbefore described, eukaryotic cells including plant cells, insect cells, mammalian cells including mammalian cell-culture adapted cells (e.g., cell lines), or any other cell amenable to the steps of encapsulation and subsequent disruption of a cell's membrane, wall or the like by those methods described herein. The Examples hereinafter describe embodiments of the invention that bacterial cells, and more particular E. coli. It is envisaged, however, that the scope of the present invention is not to be limited to just microbial cells.

All computer programs, algorithms, patent and scientific literature referred to herein is incorporated herein by reference.

So that the invention may be readily understood and put into practical effect, reference is made to the following non-limiting Examples.

### Example 1

### Encapsulated Cell Free Protein Synthesis

### Polyelectrolytes and Layer by Layer Assembly

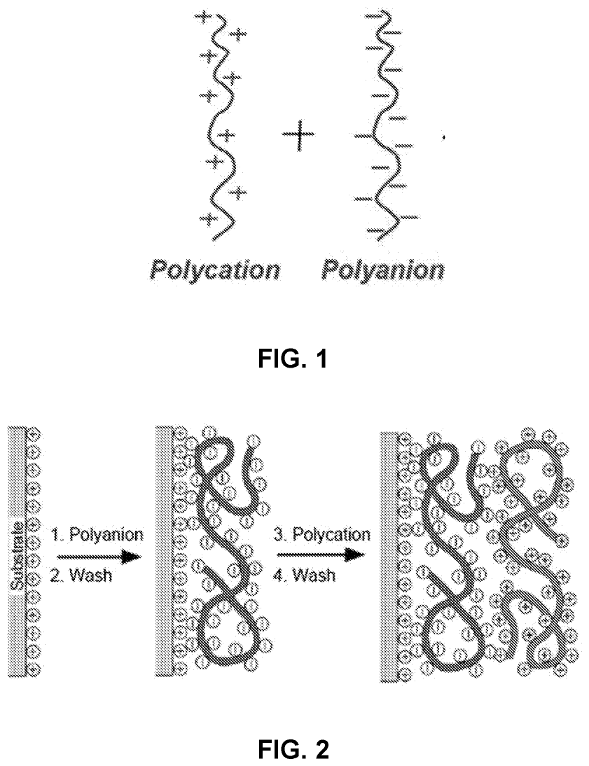

Polyelectrolytes are polymers containing electrolyte groups that disassociate in water forming a charged particle [42]. In solution polyions behave like “worm-like chains' (see FIG. 1) with the charges along the length of the polymer repelling one another [43]. Due to this natural phenomenon, the polymer itself can adsorb onto oppositely charged surfaces tightly creating a mesh like layer.

Polyelectrolytes can be used in various polymer-based assembly systems to create strongly adhering layers on any charged surface. One such method, Layer-by-Layer assembly (LbL), is a system that relies on the adsorption of long chains of polyelectrolytes on a surface, to various levels of thickness [44]. LbL mainly operates through electrostatic interactions, with high concentration of polymer in the solvent medium [45]. Various non-biological surfaces are known to have net negative charge associated through hydrolysis and surface oxidation [46]. When a negatively charged surface is exposed to a cationic based polymer, the net charge of the surface changes to positive due to adhesion and overcompensation by the polymer [46]. Washing the surface to remove loosely adsorbed polyelectrolyte leaves behind a positively charged monolayer and is ready for subsequent immersion in the negatively charged polymer solution. This is a repeat of the same procedure creates a net negative charge and a bilayer of polymer (FIG. 2). What is noteworthy of polyelectrolytes is the adsorption onto irregular surfaces, with smooth levels of uniformity [44].

Layer by layer assembly has had significant impact on the biochemical field through polymer coated nanocapsules for drug delivery, polyelectrolyte scaffolding for bone growth and cellular encapsulated library screening [47]. Often two linear polyanionic and polycationic electrolytes are used; Alginate and Chitosan respectively [48].

### Biopolymer Characteristics

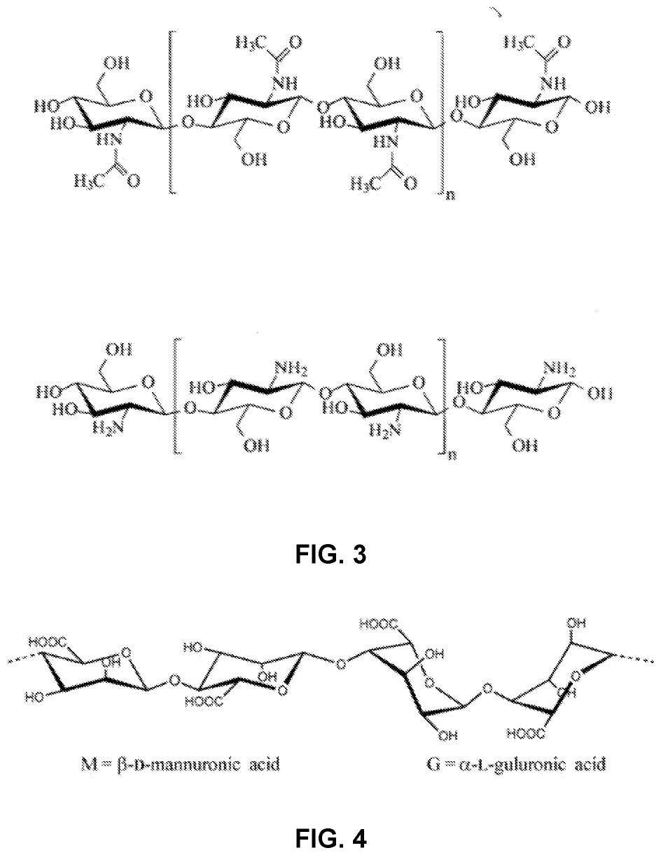

Chitin is an abundant biopolymer made up of the monomer unit 2-(acetylamino)-2-deoxy-D-glucose connected by β 1-4 linkages. It is structurally similar to D-glucose, making it comparable to cellulose [49]. It is mainly derived from the exoskeletons of arthropods such as crabs, shrimp and other crustaceans and is not soluble in water or other common solvents. Chitosan, on the other hand is soluble under certain conditions and is the deacylated form of chitin, made by treating chitin in an excess of a strong base [50]. Complete deacylation yields a simple amino group, that has a pKa of 6.5 [51]. The chemical and physical characteristics of chitosan is its hydrophilicity in dilute acidic conditions, strong cohesive interactions with anions and compatibility to a variety of biological systems [50] (see FIG. 3).

Alginic acid or Alginate in its anionic form, is a biopolymer that is used number of industries and forms a viscous gum in water [52]. It is derived from brown algae and consists of two co-polymers connected by a glycosidic 1-4 link, α-Lguluronic acid and β-D-mannuronic acid [53]. Traditionally used as a gelling agent but now has found its place in biochemistry through its strong anionic properties and adhesive capabilities [54]. Alginate has a pKa of 3.4-4.4 and is often found as an anionic salt. it readily dissociates in water forming a linear anionic polyelectrolyte which is highly adhesive to cations found in solution [53] [55] (see FIG. 4).

Escherichia coli Physical and Chemical Characteristics

Escherichia coli is a gram-negative bacterium, which has a cell wall consisting of a peptidoglycan layer with lipopolysaccharides on the surface and a phospholipid membrane [56]. Anionic groups present in these layers gives the E. coli cell wall a net negative charge and previously it has been demonstrated that LbL assembly can be utilised to encapsulate bacterial cells [57].

High internal pressure exists within E. coli, between 3-5 atmospheres and is stabilised by the highly crosslinked peptidoglycan layer [58]. Theoretically, only a small number of these crosslinks need to be sheared for the plasma membrane to be ruptured and cause hypotonic lysis. It has been suggested that a single endolysin molecule which specifically cleaves peptidoglycan bonds, could theoretically cleave enough of the peptidoglycan layer to kill a bacterium [59].

Lysin Mediated Autolysis of E. coli

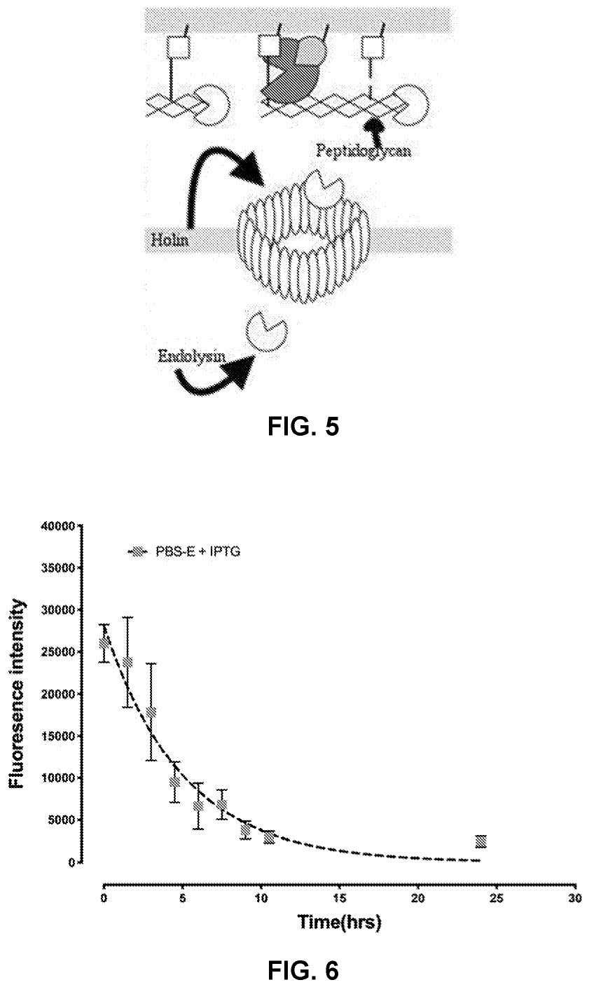

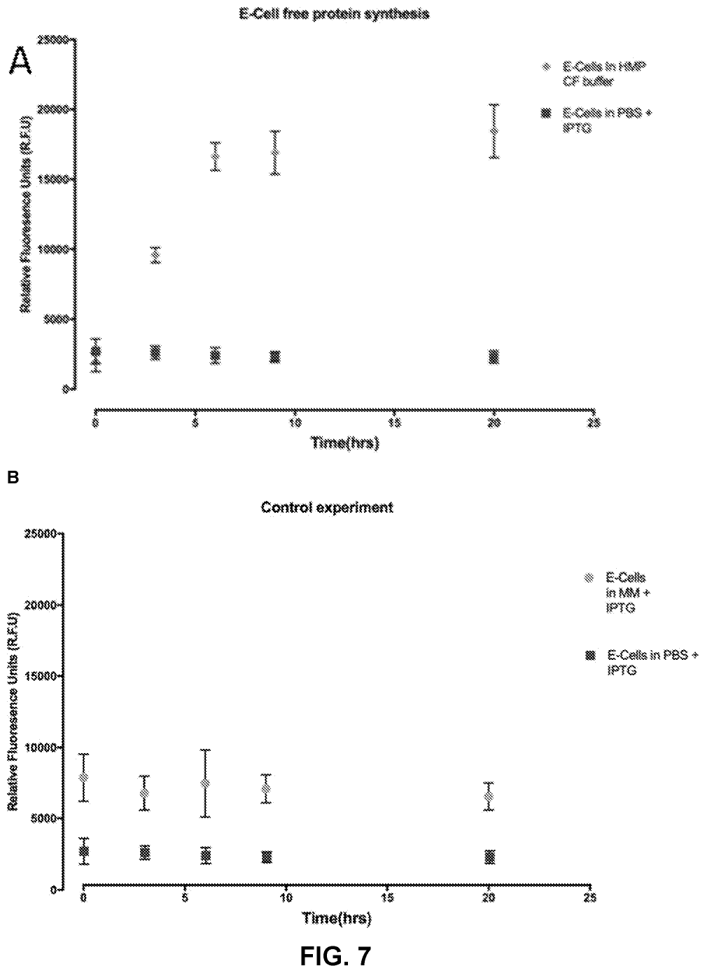

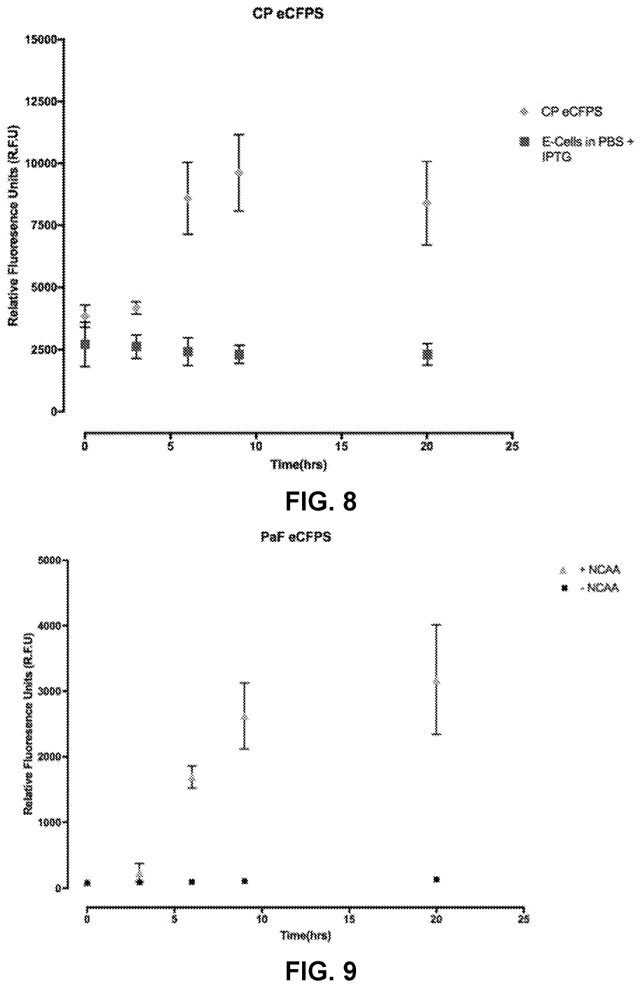

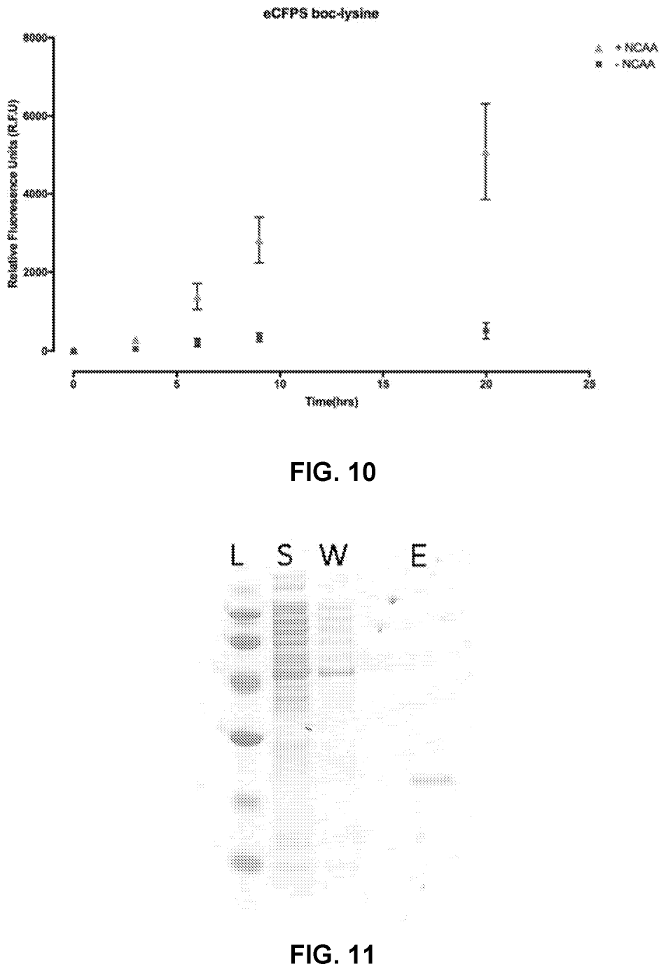

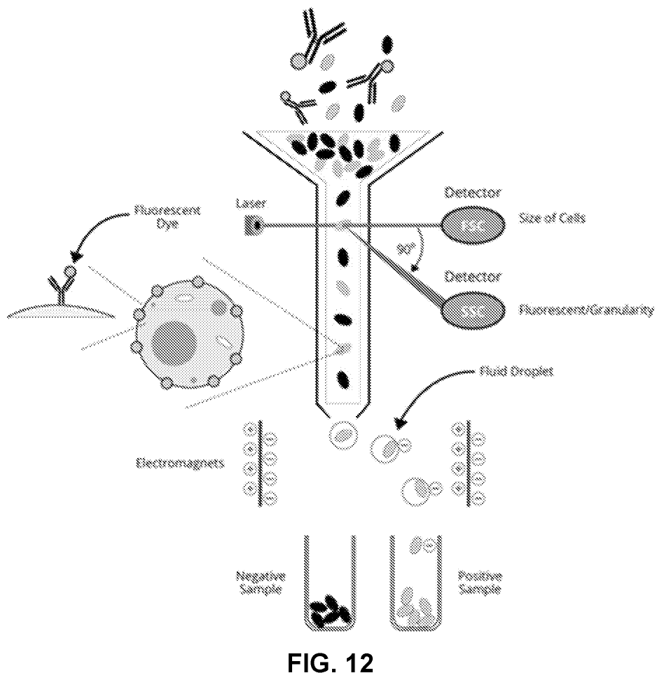

Endolysin is a protein produced from bacterium infected by bacteriophages in their lytic cycle and requires an accessory protein to function. These proteins are termed ‘holins’ and they are used to create a ‘holin-mediated permeabilising event’ or ‘hole’ in the phospholipid membrane and allow for endolysin to have access to its peptidoglycan substrate (see FIG. 5) [60]. The Xjb(DE3)* autolysis cell line is a derivative of BL21(DE3)*2 and expresses endolysin that is encoded in the genome under an arabinose inducible pBAD promoter [61]. Xjb cells require a freeze-thaw step as holins are not encoded to create a permeabilising event. The freeze-thaw disrupts the plasma membrane and creates physical holes that allow endolysin to reach the peptidoglycan layer and cleave bonds to cause lysis [62].

LbL assembly can be applied to the Xjb autolysis strain after induction with arabinose. Endolysin will build up in the cytosol and can be autolysed after the LbL process. The encapsulation is done in the log phase of growth to facilitate the most efficient content of transcription and translation equipment. The final product is a permeable membrane where small molecules can diffuse freely, while macromolecules are retained. Plasmids, translation and transcription equipment remain in this encapsulated shell, as endolysin does not have the capability to cleave polymer bonds.

### Experimental Pore Size Validated by Diffusion

To understand whether these capsules actually confined macromolecules, an expression plasmid with a reporter gene (722WT mNeonGreenGFP) was expressed in the Xjb cell line and then encapsulated using chitosan/alginate. Based on this 26.9 kDa variant of GFP, the passive diffusion of macromolecules from the encapsulation can be modelled by nGFP diffusion from the bilayer.

Rapid diffusion of nGFP occurs over the course of six hours and then remains static. Of the 100% of nGFP present at the beginning of diffusion, only 10% is present 20 hours later. The half point of diffusion occurs at 4 hours, meaning that most macromolecules are present in some quantity at this point. The large majority of CFPS occurs within the first 6 hours and theoretically it is possible to induce the encapsulated cells for protein expression as the essential protein synthesis equipment is present. Large components of translation and transcription greater than 26.9 kDa will diffuse out of the semi-permeable membrane much more slowly and large elements would be retained for a longer time, furthering the possibility of CFPS. Active nGFP diffusion substantiates that lysis has occurred, as proteins in live cells are not able to diffuse protein, which means that a permeable bilayer for CFPS is present.

### Encapsulated Cell Free-Protein Synthesis

To test whether these semi-permeable encapsulated cells could sustain protein synthesis, the following experiment was conducted. LB was used to grow a Xjb cell with a 722WT mNeonGreenGFP as a reporter gene with 3 mM arabinose present to express endolysin. Chitosan/alginate encapsulation was conducted at culture growth of 0.6 OD to retain efficient content of transcription and translation components. This yielded an encapsulated Xjb cell line containing uninduced nGFP and was subsequently freeze-thawed for lysis. The lysed capsules were immediately immersed in CFPS buffer based on the ATP regeneration method using Maltodextrin/hexametaphosphate (HMP/MD) and protease inhibitor (PI).

The results indicate that the capsules produced nGFP over 20 hours, meaning that this is the first reported encapsulated system that can express recombinant protein. All the enzymes necessary for translation, transcription and energy generation are present within this ‘Encapsulated cell free system’ and by coupling this with the HMP/MD CFPS energy generation method, reduces in-vitro expression cost dramatically.

This method can be considered analogous to other in vitro CFPS systems with the numerous advantages that are associated with CFPS. The chemical environment of the E-cells can be altered to fit the desired experimental conditions and reaction can be scaled down.

By back-calculating from the calibration curve and its line equation, the estimate of final protein concentration from the HMP encapsulated system was approximately 0.045 mM at 20 hours. There is an offset of fluorescence, as the WT system uses a loosely regulated T7 RNAP which leads to a small amount of leaky expression. Approximately 0.004 mM nGFP was already present before induction with CFPS buffer, although this can be controlled by using a more tightly regulated promoter. Although this is the case, there is an obvious difference between the induced CFPS system and control, with more than ten times increase in GFP fluorescence. This indicates that encapsulated CFPS was successful and that the system can be induced as an in vitro method. The volume of the reaction buffer was 500 μl showing that protein production encapsulated CFPS retains the ability to be conducted in a small reaction volume as with other CFPS methods.

WT E-cells give a strong background reading of fluorescence that needs to be corrected for using uninduced cells. This is in contrast to standard CFPS, where there is no fluorescence for the uninduced WT system and standard CFPS controls have no fluorescence over time.

The second graph consists of a control experiment, which was used to determine whether these E-cells still retained in-vivo activity. The WT nGFP E-cells were incubated in GMML and induced with 1 mM IPTG to determine whether they still were able to produce protein. GMML has all the components necessary for cellular metabolism and GFP production would occur in live cells. As the graph indicates, there was small decrease in GFP content demonstrating that protein production was non-existent. Thus E-cells have no in-vivo activity, providing evidence that they are a strictly in-vitro system.

S30 is approximately 20 times more dilute than in the cell, which decreases rates of elongation, initiation and protein synthesis as a whole [7]. Conversely in E-cells, all of the transcription and translation elements are confined closely in individual membrane bags and are in higher concentration than in a standard S30 extract. This is why expression in E-cells works efficiently as the unfettered access that transcription/translation proteins have to one another in an encapsulated CFPS system is more capable than in standard CFPS systems.

Some ancillary processes assist protein translation and identifying these specific proteins that do so may be difficult to quantify in the S30 extract. For example, the PURE system consists of reconstituted proteins directly related to protein transcription/translation, individually purified and used for protein expression [63]. There was quite a low level of expression which is indicative of a complex network of proteins involved in protein production [28]. E-cells hold the entire repertoire of protein elements as an in-vivo system and after lysis they are held within the encapsulation. This can provide added efficiency to multi-enzymatic in-vitro systems that utilise membrane proteins.

E. coli polyphosphate kinases (PPK) are outer membrane bound proteins that facilitate the synthesis and degradation of linear polymers of energy rich polyphosphate molecules [64]. The ATP regeneration method utilising maltodextrin and HMP is based on the acquisition of phosphate molecules from a linear phosphate. In the results below, the reaction capabilities of these ‘E-cells’ are much higher than compared with the same ATP regeneration system in batch CFPS. In chapter two the soluble fraction of E. coli was utilised in batch CFPS, which may have handicapped the ATP regeneration system as some PPK is lost during S30 preparation. There have been efforts to circumvent this issue by supplementing heat treated E. coli cells that have overexpressed thermophilic PPK directly into the ATP regeneration system with success [65]. This completeness of the protein repertoire confers significant advantage onto encapsulated CFPS, as protein networks that are not present in standard CFPS are present in encapsulated CFPS.

One of the major advantages of this system is that it does not require an S30 extract, which is one of the most time-consuming and costly processes of CFPS reagent preparation [4]. Methods of early S30 preparation are known to take an extensive amount of manpower to prepare. It recent years, S30 has been restructured so that certain unnecessary time-consuming steps are excluded. Although S30 extract preparation has been streamlined, it still requires cell breakage, washing with various buffers and centrifugation at high speeds which are time and cost intensive.

Conversely E-cell preparation can be completed within a timeframe of 2 hours after culture has been grown to a specific optical density(OD) and then used immediately afterwards. Inexpensive reagents are used for the LbL assembly and encapsulated cells can be stored indefinitely. When these encapsulated cells are made, they suit an individual purpose as plasmids are held within the encapsulation, but the speed in which they can be prepared presents significant advantage over other methods.

Encapsulated CFPS was conducted using a conventional system of protein expression, CP and the exogenous enzyme Creatine kinase. The purpose of this experiment was to demonstrate whether a common eukaryotic system of energy generation can be applied to these cells. 500 μl of the revised CP/CK CFPS buffer was added to this system using uninduced WT 722 nGFP encapsulated cells and PI was added.

### Encapsulated CFPS Comparison

The yield of the CP system was 0.02 mM with approximately half of that being background fluorescence (0.0088 mM) outlining the need for a tightly regulated promoter. There is clear production of nGFP from encapsulated CFPS using the CP system, although it is not as distinct in comparison to the HMP system. This is probably due to the build-up of free phosphate when using the CP/CK system and this is indicated by the short length of the reaction. Conversely, when using HMP there is an extended amount of protein production over 20 hours, most likely due to the ability of the system to regenerate free phosphate. The regeneration of Pᵢ may be enhanced with the use of the HMP method, as the membrane associated PPK is most likely present within the ‘E-cells’ at a higher quantity than S30.

### Encapsulated CFPS and NCAA Incorporation

This novel method can be considered an equivalent to a synthetic cell as it has a chimeric duality between the characteristics of an in vitro and an in vivo system. Unlike standard CFPS methods auxiliary proteins required for NCAA incorporation do not need to be supplemented in the reaction mixture. From the results above ‘E-Cells’ have demonstrated their permeability and it is logical to introduce this system as a step forward in NCAA incorporation. The first test that was conducted to demonstrate possible NCAA incorporation was the use of a Mj. TyrRS synthetase selected for p-acetyl-L-phenylalanine [14]. This synthetase was selected for by Schultz et al in 2004 and using pCDF amber nGFP as a reporter protein NCAA incorporation was attempted using encapsulated CFPS.

### Results and Discussion of PaF Incorporation

Amber pCDF nGFP was used as a reporter protein in conjunction with a p-acetyl-L-phenylalanine synthetase/tRNAₛᵤₚ. The experiment was conducted with 500 μl of CFPS buffer using the HMP and maltodextrin method of energy generation with the addition of PI. There was only one control used and it had identical composition apart from the absence of NCAA.

In the results, there is a distinct difference between the + and − NCAA in terms of fluorescence, indicating that successful incorporation of p-acetyl-L-phenylalanine had occurred. The control remained non-fluorescent which demonstrates clear NCAA specificity of the aaRS. There is no nAA suppression of the amber stop codon which has been confirmed in the literature. The reported amber suppression in [14] was exclusively PaF with a reported fidelity of 99.8%, which is validated by the experimental data above.

This experiment confirms the ability for NCAAs to be incorporated into an encapsulated CFPS system. As there was no synthetase or tRNA supplemented with in the CFPS reaction, it highlights the robustness of this system as the tRNA/aaRS is expressed during growth. There is no need to express and then purify tRNA/aaRS which is required in conventional in vitro systems and the speed at which encapsulation is achieved allows for a tremendous reduction in labour. After a dual transformation of aaRS/tRNA pair and reporter gene, an experimenter could theoretically start CFPS the same day as inoculation, without the need for the long HIS-tagged aaRS purification step. Encapsulated CFPS provides a platform where experimenters can easily perform NCAA incorporation and streamlines in vitro expression of NCAA modified proteins immensely.

Methanosarcia mezei PylRS boc-Lysine Incorporation

aaRS/tRNAₛᵤₚ purification process for PylRS addition into CFPS is complex, complicating NCAA incorporation drastically [66]. More concerning, the purified components can only be stored for a relatively short period of time before becoming inactive [66]. This is a severe limitation that is placed onto scientists hoping to achieve NCAA incorporation in an in vitro setting.

A previously described method to circumvent this complex purification was by creating a S30 extract transformed with the cognate aaRS/tRNAₛᵤₚ pair [66]. A culture expressing the aaRS/tRNAₛᵤₚ was grown for ˜13 hours and then the S30 extract was prepared. This was successful in a standard CFPS setting and allowed for NCAA incorporation in a PylRS system. A similar experiment was attempted in the Huber/Otting group, but PylRS would aggregate in the S30 extract, preventing its use for CFPS.

Pyrrolysl-RS(PylRS) and its derivatives are known to incorporate at least 101 NCAAs, which is a staggering amount and constitutes 52% of the NCAA library [66]. Purifying PylRS is extremely convoluted and it is rarely used as a purified component in an CFPS setting. Most of PylRS NCAAs that are incorporated come from an in-vivo setting, which is expensive due to the cost of NCAA [24].

Using an established example of PylRS incorporation we demonstrate the ease of which a traditionally difficult NCAA incorporation can be achieved using encapsulated CFPS. This experiment is based on the work where Yokoyama et al previously engineered a Methanosarcina mazei PylRS/tRNAₛᵤₚ pair for N□-(tert-butoxycarbonyl)-L-lysine (boc-lysine) [67]. Xjb cells were transformed with a pCDF GFP vector and an aaRS/tRNAₛᵤₚ and then grown to OD 0.6 and encapsulated. The cells were lysed and then induced with 500 μl MD/HMP based CFPS buffer and PI.

### Results and Discussion

The results show a clear separation between the + and − NCAA fluorescence indicating that there was successful incorporation of boc-lysine with encapsulated CFPS. There seems to be some level of nAA suppression in the control experiment and this is confirmed in the literature. There is a mutation from A-G on the tRNAₛᵤₚ acceptor stem that decreases the NCAA suppression efficiency by 42% and increases nAA suppression [68].

It is easily understood how difficult protein expression using in vitro NCAA incorporation can be circumvented with encapsulated CFPS. This highlights the robustness of this platform for NCAA incorporation and how it still retains the cost-effective scale of CFPS. As little as 15 μl of 100 mM stock of NCAA is used in this experiment, which also demonstrates the fast screening capabilities of encapsulated CFPS. This coupled with the redundancy of supplementing aaRS/tRNAₛᵤₚ makes this a quick and easy tool for NCAA incorporation.

To confirm that incorporation occurred successfully, the first large scale encapsulation and encapsulated CFPS was conducted in 3 ml of MD/HMP CFPS buffer, using 6×HIS tagged amber Peptidyl-prolyl cis-trans isomerase B (PPiB) as a reporter protein. Purification and then mass spectrometry was then conducted to identify whether incorporation had occurred.

There was good expression of PPiB as outlined by the gel with a yield of 0.048 mM in 500 μl. the economical use of NCAA can be highlighted as 30 μl of 100 mM boc-lysine was supplemented into HMP encapsulated CFPS system. This reduces the cost of the experiment by orders of magnitude with a cost-effective lysate, CFPS buffer and NCAA usage.

The mass spectrometry results confirmed the presence of boc-lysine in PPiB (MS data can be found in supplementary info). The m/z for 6×HIS amber PPiB without any suppression of the amber site is 18839.18, the two masses that are most notable is obviously the peak at 19066.41 which corresponds to boc-lysine incorporated PPiB. The second is the small peak at 18966 which corresponds to the natural suppression of lysine or glutamate. tRNA_{glu} has an anti-codon of CUU which is almost complimentary to the amber stop codon (CUA) and is the most likely cause for the natural suppression. The nAA suppression compared with the boc-lysine peak is comparatively small, with the majority of protein being boc-lysine incorporated at a site specific amber region. The other peak at 19023 may correspond to a tryptophan residue as the MW of Trp is 204 which is 0.63 Da short. The peak at 19050 does not correspond to any known amino acid, although it may be an adduct of boc-lysine.

This was one of the few times that in-vitro incorporation of boc-lysine using a PylRS has occurred and the first time that an encapsulated system produced recombinant protein on such a large scale. The implications of this method onto the biochemical field is large with a wide variety of applications for encapsulated CFPS.

### Example 2

### Selection Systems and Encapsulated Cells

The robustness of the E-cells is not limited to simply circumventing purification methods, but theoretically allows for toxic and membrane-impermeable NCAAs to be incorporated. Phospho-tyrosine is an NCAA that has issues crossing the negatively charged E. coli cell wall. Using the permeability of E-cells, a library selection system based on GFP fluorescence was constructed for the non-hydrolysable mimetic of phosphotyrosine, 4-phosphono-methyl-L-phenylalanine [69]. Positive selection was applied to capsules harbouring plasmids that encode for functional tRNA synthetases. Positive selection occurred through a library of variants which was co-transformed with the reporter gene amber GFP. The cells were lysed and encapsulated CFPS was subsequently performed.

During encapsulated CFPS only functional aaRS/tRNAsup pairs will be able to incorporate NCAAs or nAAs and allow for full length incorporation. The positive pairs will fluoresce allowing encapsulated cells to be distinguished based on their fluorescence and sorted using Fluorescence activated cell sorting (FACS). After iterative rounds of selection, the plasmids can be isolated and provide experimenters with functionally incorporating aaRS/tRNAₛᵤₚ.

### Introduction to Fluorescence Activated Cell Sorting

Fluorescence activated cell sorting or (FACS) is a derivative of flow cytometry, which allows for sorting of heterogeneous cell culture into separate containers utilising the physical dimensions of light scattering [70]. Cells are sorted based on their physical properties i.e. how light scatters when it passes through a cell and its fluorescence. If a cell possesses the experimentally defined characteristics it is sorted as positive and if negative it is removed from the sorted sample.

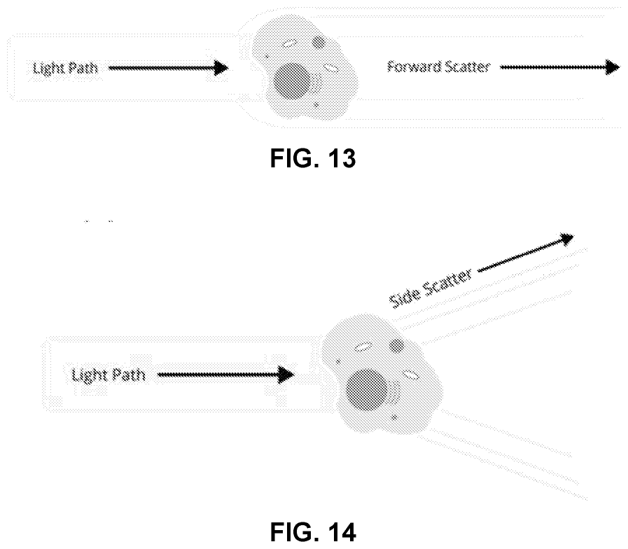

There are three main features of FACS that determine the physical characteristics of a cell. The first is fluorescence and can be sorted based on its emission intensity after chromophore excitation. The other two are termed ‘forward scatter’ and ‘side scatter’ and both parameters provide experimenters the means to sort cells [7]. Low angle-forward scatter is the light refracted by the cell in a forward direction continuing through the cell and determines the size [71]. Oftentimes this light is taken up to a 20° angle and larger cells will produce more forward scatter (see FIG. 13).

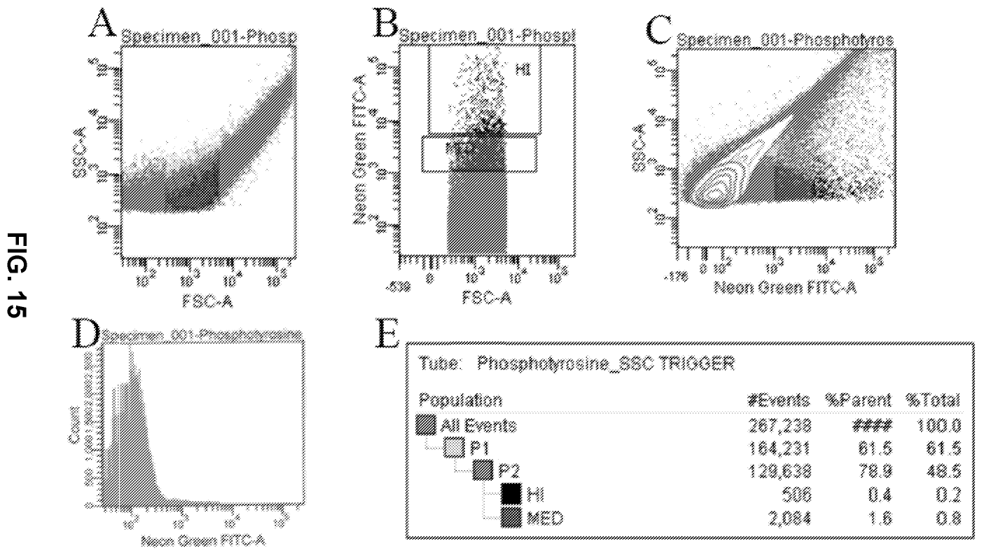

Side scatter is the light refracted from cells that does not continue in the forward direction, but to the sides, often measured at the 90o perpendicular to the excitation line [70]. It provides information on the complexity of cells, as cells with a higher internal complexity will scatter more light to the side (see FIG. 14).

PmP and pOpt Library Characteristics

Using these characteristics, a library based on p-cyanophenylalanine tRNA synthetase derived from a Mj. TyrRS synthetase was sorted. The library is termed pOpt and consists of a randomised set of 6 residues in the active site based on the crystal structure of p-CNF-RS. Tyr32, Val65, Met109, Ala159, Leu162 were completely randomised. The remaining residue Gly158 was mutated to a mixture of Gly and Ser which decreases Tyr affinity as a substrate. The library is in a pBK backbone under a GlnS constitutive promoter/terminator which contains a cognate optimised suppressor tRNA, with the final count of possible aaRS mutants to be 6.4×10⁶.

Based on the physical characteristics: the SSC, FSC and fluorescence of these capsules, we can distinguish between the non-fluorescent aggregate capsules and individual ‘E-cells’. This is important as aggregated capsules may have more than one plasmid encoded and if one is non-functional when it is sorted, it contaminates the pool of possibly functional synthetases.

### Results of First Round PmP FACS

The library was encapsulated using a pCDF amber GFP under an IPTG promoter in the Xjb cell line. Cells were subsequently encapsulated and encapsulated-CFPS was performed using a non-hydrolysable analogue of phospho-tyrosine, 4-phosphono-methyl-L-phenylalanine (PmP) for positive selection. There was clear fluorescent difference between the control and PmP focused library indicating that encapsulated CFPS was successful. The encapsulated product was placed in a FACS machine and sorted to single capsules.

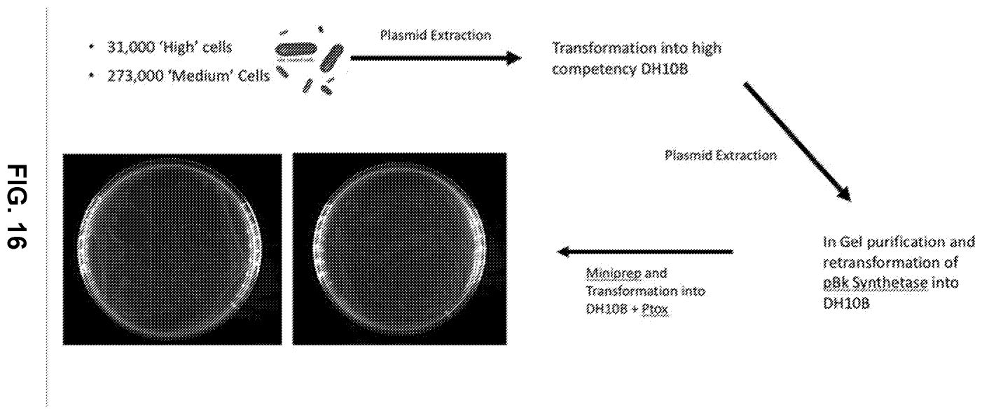

The gates for sorting were selected based on the individual microcapsules sorting from previous literature [72]. From the diversity of fluorescence levels there seemed to be an assortment of aaRS mutants, which corresponds to library diversity. In addition, the relatively small number of fluorescent compared to non-fluorescent aaRS shows a small amount of sequence space that incorporate can actually incorporate PmP, as seen in FIGS. 15 (D & E) with only 2% of fluorescent single particles. There may even be redundancy between aaRS populations in the positive E-cells, with the pool having more than one of the same aaRS.

Positive selection included sorting the fluorescent capsules from non-fluorescent capsules. There were two lots of encapsulated cells that were sorted designated PmP high and medium, which were labelled based on their fluorescence level. 273,000 cells of PmP med and 31,000 high were sorted into separate tubes and the plasmids were extracted using light sonication and a PCR cleanup to remove salts.

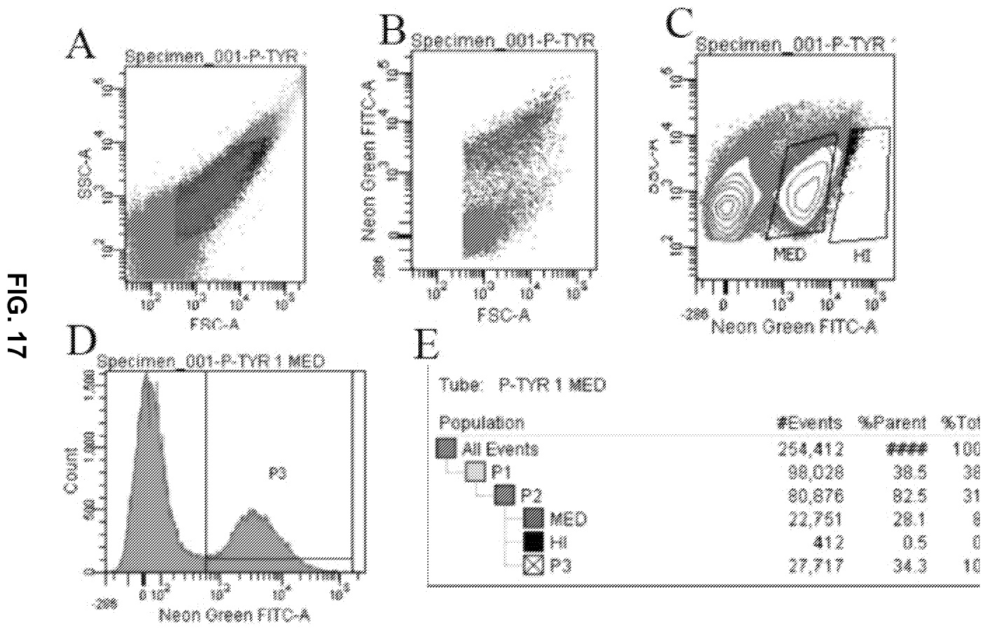

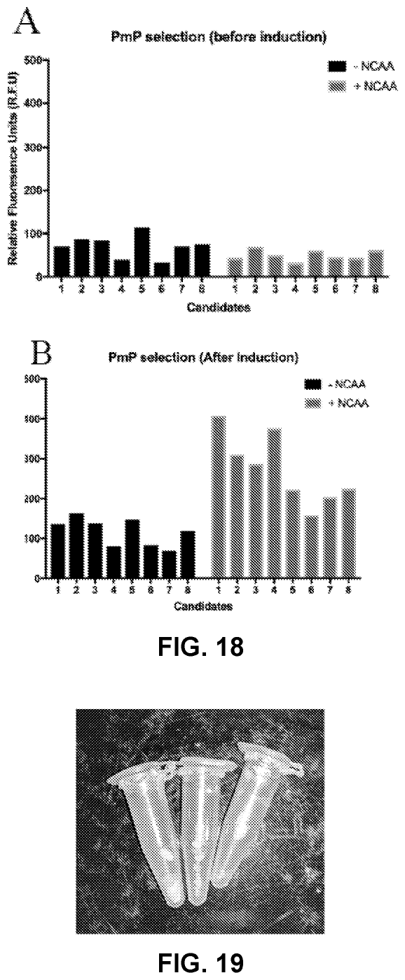

After positive selection, a traditional plate-based system was used for negative selection. Amber interrupted barnase transformed into DH10B cells was used for negative selection. The aaRS library was simultaneously transformed with amber barnase and grown on arabinose plates which induces amber barnase expression for nAA incorporating mutants. There was substantial cell growth on each of the plates indicating that negative selection was successful and no nAA was incorporated (full schematic in FIG. 16). The cells were harvested and retransformed into Xjb. Encapsulated CFPS was performed and then the cells were sorted again via FACS to determine whether positive selection was successfully achieved through an increase of a defined fluorescent population.

### Second Round of PmP FACS

The second round of positive selection showed an enrichment of library aaRS and is indicative that the FACS positive selection is operational. The proportion of fluorescent capsules increased from 1.6% in the total library to 28.6% in the second round of positive selection, this demonstrates that there is discrimination towards mutants that can suppress amber mutations. In FIGS. 17(B,C and D) there was clear distinction between fluorescent and non-fluorescent populations, that did not exist in previous round of selection, which indicates that selection rounds were effective.

This fully demonstrates that iterations of positive selection can occur using FACS and encapsulated cells. Isolating each of these mutants is the next step to find a functional aaRS pair that can incorporate PmP.

### Isolation of PmP Candidates

After sorting 8 million encapsulated cells for the second round of PmP E-cells, the same procedure was conducted after the first sorting. The cells were transformed into pTox DH10B, which codes for amber barnase to remove the nAA incorporating aaRS from the library. Afterwards the selected library was transformed into DH10B to identify and select single mutants. The mutants were given a number according to the order they were picked, from 1-8 and then they were encapsulated with amber pCDF GFP in Xjb individually. 500 μl of MD/HMP CF buffer was used for encapsulated CFPS and a + and − NCAA control was established with each reaction mixture being identical (apart from NCAA).

Each candidate shows a clear separation between + NCAA and − NCAA meaning that the selection rounds resulted in aaRS that had NCAA specificity. Auto-fluorescence or natural emission of light is also present in the E-cells to an extent. Offset by auto-fluorescence increases the + and − NCAA fluorescence after induction which is seen in FIG. 17 (A to B). The offset is heightened, as the GlnS promoter is weak and expresses the aaRS/tRNAₛᵤₚ pair poorly. This leads to reduced amber suppression and causes a low fluorescence value which is affected more by the offset.

There seems to some level of nAA suppression in the various candidates. Number 7, seemed to give negligible nAA incorporation and remained at the same fluorescence before and after induction for the control. Overall the E-cell selection resulted in the isolation of individual synthetases that incorporate the membrane-impermeable PmP.

### Encapsulated Cell-Free Protein Synthesis Summary

A novel, highly cost-effective platform for in vitro translation has been developed reducing the cost of S30 extract preparation and purification of ancillary proteins. This has the potential to revolutionise CFPS as the ease of setup and inexpensive reagents allows unrestricted access amongst almost all laboratories that utilise CFPS. It is highly versatile and can be used with conventional and non-conventional energy generation systems furthering its accessibility as a platform. Alginate and Chitosan are inexpensive and only minute amounts are used during the encapsulation procedure compared with the lengthy procedure and buffers used for S30.

The encapsulation and use of lysed cells in-vitro circumvents issues with proteins that are difficult to purify, as is the case with PylRS. Reducing and oxidising conditions can be introduced to encapsulated system for proteins that require a certain environment to function.

The high level of expression of encapsulated CFPS makes it competitive to other CFPS systems, especially when cost is taken into consideration. Its individualistic driven design offers flexibility to suit individual researchers own needs and provides significant advantage over more conventional methods. Simple purification attributed to most CFPS systems is still attached to encapsulated CFPS as seen with the purification of boc-lysine incorporated PPiB.

### Selection and CFPS

There has been significant progress in a selection system utilising ‘E-cells’ in a selection system based on p-cyanophenylalanine RS. Isolation of aaRS plasmids using iterative rounds of selection indicates that functional synthetases have been found to incorporate PmP. The speed at which this selection system can be implemented has to be noted. Traditional plate-based selection is an extremely time-consuming process, and the use of FACS significantly reduces the time spent plating individual candidates, which is convention when using CAT as positive selection.

Batch CFPS has been developed sufficiently well to be used as a lab wide practice. It is comparable to dialysis (lab standard) when there is no PI and greatly exceeds expected yields when PI introduced. The economic use of reagents in batch CFPS warrants its use over dialysis as the preferred system, as dialysis has an outside buffer that is made up to 10× the inner buffer and is often >2 ml in quantity. Batch CFPS can be used from the scale of 100 μl up to mL quantities with the same or higher protein yield. Batch CFPS does not require a dialysis membrane to function which allows for high-throughput screening of proteins, as tying individual dialysis knots for innumerable aaRS candidates is extremely labour-intensive.

### Future Direction

Lyophilising E-Cells and inducing them using CFPS is the next innovation for portable protein translation. As it has been demonstrated, E-Cells are a portable CFPS platform where large quantities of proteins can be produced very quickly and if applied in a lyophilised format has many applications.

There has already been progress in the lyophilising of S30 extract with certain lyo-protecting groups being introduced to retain expression activity. Sucrose is used as a lyo-protecting group and it has been shown to stabilise lyophilised S30 extracts for an extended period of time [73]. This can be feasibly applied to E-cells as the same components that are present in the S30 are present in the E-cells. There is the advantage of plasmid already being ready for translation and requires only the CFPS buffer to induce expression. Progress has also been made lyophilising CFPS buffer for extended storage periods and combining this with E-cells makes the first portable in-vitro expression platform available. Quick synthesis of therapeutic proteins can be achieved using this portable system, with on-the-go in vitro translation being possible. It can be applied to personnel in remote locations without the full access of a research lab the components can be pre-prepared. This has applications with a plethora of industries such as defence, pharmaceutical industries, research and medical related fields.

Further isolation of aaRS mutants using toxic and hard to incorporate NCAA is the next step for E-Cell selection. Dipicolinic acid or DPA selection is currently being used in ‘E-cell’ system and is the next step in our selection process of function aaRS.

### Materials and Methods

General Laboratory Methods ie—Plasmid Construction, Sequencing, PCR, Vector Extraction

Oligonucleotides and primers were obtained from IDT technologies for cloning reactions, in house E2, DpN1, Q5 and Vent were used for cloning reactions. BigDye terminator for sanger sequencing was obtained from Thermo Scientific and PCR products were purified using ethanol/EDTA precipitation. pCDF amber GFP was constructed from pCDF amber barnase and 722 amber nGFP both obtained from members of the Huber lab. Transformation was primarily done with highly electro competent cells, grown with SOB media and washed with ice cold 10% glycerol twice, then a Bio Rad MicroPulser™ was used.

### Protein Purification, Mass Spectrometry

1 ml His GraviTrap TALON columns (GE Healthcare) were used for the purification of PPiB, which was expressed in an encapsulated pellet from 500 ml of culture, 3 mls of CFPS to induce expression and had approximately 0.5 mg/ml of boc-lysine incorporated PPiB in 500 μl. Mass spectrometry was based on ESI-MS with an Orbitrap Elite mass spectrometer (Thermo Scientific), and mass spectra was processed using Xcalibur software package.

### Encapsulation Procedure

Xjb cells were used and induced at the beginning of inoculation with a final concentration of 3 mM arabinose. The cells were grown to OD 0.6 and washed three times with PBS-E pH 7.4 and resuspended in 0.25 mg/ml of Chitosan in PBS-E solution with vigorous shaking for 20 minutes. The cell pellet was washed with PBS-E pH 6.0 three times and then resuspended in 0.25 mg/ml of Alginate PBS-E solution and subjected to vigorous shaking for 20 minutes. The cells were washed 3 times with PBS-E pH 6.0 and then resuspended in PBS-E pH 7.4 and stored at −80o centigrade. Recipes for reagents can be found in the supplementary information. CFPS was induced after thawing, using a revised procedure that can be found in the supplementary information.

### CFPS and Fluorescence Readings

Conducted at 37° C. in an induction shaker at 180 rpm, fluorescence readings were taken every three hours using Softmax pro. nGFP has an excitation of 506 and an emission of 517, but the error on the machine is approximately 10-15 nM. A revised measurement was used to read a part of the emitted light at 560 nm which was far from the error of the machine.

Three recipes for CFPS were used:

(1) HMP/Maltodextrin CFPS recipe contained 0.9 mM of UTP and CTP, 50 mM HEPES, 1.5 mM GTP, 1.5 mM ATP, 0.68 uM folinic acid, 0.64 mM cAMP, 1.7 mM DTT, 3.5 mM of AA mix (see [4] for detailed recipe), KGlu 60 mM, MgGlu 6 mM, 2% v/v PEG-8000, 5 mM CoA, 35 mM Maltodextrin, 30 mM 3-PGA, 1.2 mM HMP and 10 mM NAD. S30 made up 30% of reaction volume and Roche complete Mini™ PI inhibitor was added to the master mix after being dissolved in 5 mL. IPTG and NCAAs were at 1 mM when used in an encapsulated CFPS setting. NCAA incorporation was done with 1 mM final concentration of NCAA in HMP/MD CFPS buffer.

(2) CP/CK CFPS contained 0.9 mM of UTP and CTP, 50 mM HEPES, 1.5 mM GTP, 1.5 mM ATP, 0.68 uM folinic acid, 0.64 mM cAMP, 1.7 mM DTT, 3.5 mM of AA mix (see [4] for detailed recipe), KGlu 60 mM, MgGlu 6 mM, CP 80 mM and CK 250 ug/ml. S30 made up 30% of the reaction volume and Roche complete Mini™ PI inhibitor was added to the master mix after being dissolved in 5 mL.

(3) Dialysis mode based on [4] was 0.8 mM rNTP (UTP, GTP, CTP), HEPES 55 mM, ATP 1.2 mM, folinic acid 0.68 um, 0.64 mM cAMP, 1.7 mM DTT, 27.5 mM ammonium acetate, 1 mM AA mix, 208 mM KGlu, Mg(OAc)₂ 15 mM inner/19.3 mM outer, CP 80 mM and 250 ug/ml CK. s30 was 30% of the final reaction mixture. the inner buffer was placed in a 10-15 kDA cutoff cellulose dialysis membrane and the outer buffer was made up to 10× the inner buffer.

Selection and FACS: pOpt library was obtained from Abera Saeed's work in the previous honors cohort and was transformed into the Xjb(DE3)* cell line with a compatible reporter gene, pCDF amber nGFP. The library was then induced with CFPS buffer and sorted.

FACSdiva was used and samples were submitted to Harpreet Vohra at JCMS for sorting and analysis. Graphs were generated at JCMS.

There were two rounds of negative selection and they consisted of extracting aaRS plasmid from sorted E-cells. The sorted cell and their plasmids were extracted using a light sonic bath and the solution was freeze dried. PCR clean-up was used to remove the excess salt in the freeze-dried sample. The plasmid was retransformed into DH10B and then extracting the plasmid for amplification of the selected library. Gel purification followed to remove pCDF amber GFP and then transformed into DH10B again to reamplify plasmid. The plasmid was extracted and then transformed into pTox housing pBAD amber barnase and then plated onto arabinose plates. Colonies that were grown were harvested in totality and then the plasmid was gel purified to remove pTox. The selected aaRS library was transformed into DH10B and colonies were picked individually as single aaRS mutants.

### REFERENCES

[1] F. Baneyx, “Recombinant protein expression in Escherichia coli,” Curr. Opin. Biotechnol., vol. 10, no. 5, pp. 411-421, October 1999.
[2] W. C. Yang, K. G. Patel, H. E. Wong, and J. R. Swartz, “Simplifying and streamlining Escherichia coli-based cell-free protein synthesis,” Biotechnol. Prog., vol. 28, no. 2, pp. 413-420, March 2012.
[3] Q. Lian, H. Cao, and F. Wang, “The Cost-Efficiency Realization in the Escherichia coli-Based Cell-Free Protein Synthesis Systems,” Appl. Biochem. Biotechnol., vol. 174, no. 7, pp. 2351-2367, December 2014.
[4] M. A. Apponyi, K. Ozawa, N. E. Dixon, and G. Otting, “Cell-Free Protein Synthesis for Analysis by NMR Spectroscopy,” in Methods in molecular biology (Clifton, N.J.), vol. 426,2008, pp. 257-268.
[5] D. Foshag et al., “The E. coli S30 lysate proteome: A prototype for cell-free protein production,” N. Biotechnol., vol. 40, no. Pt B, pp. 245-260, January 2018.
[6] E. D. Carlson, R. Gan, C. E. Hodgman, and M. C. Jewett, “Cell-free protein synthesis: Applications come of age,” Biotechnol. Adv., vol. 30, no. 5, pp. 1185-1194, September 2012.
[7] G. Rosenblum and B. S. Cooperman, “Engine out of the chassis: Cell-free protein synthesis and its uses,” FEBS Lett., vol. 588, pp. 261-268, 2014.
[8] F. Katzen, G. Chang, and W. Kudlicki, “The past, present and future of cell-free protein synthesis,” Trends Biotechnol., vol. 23, no. 3, pp. 150-156, March 2005.
[9] T. S. Young and P. G. Schultz, “Beyond the Canonical 20 Amino Acids: Expanding the Genetic Lexicon *,” 2010.
[10] A. Ambrogelly, S. Palioura, and D. Soll, “Natural expansion of the genetic code,” Nat. Chem. Biol., vol. 3, no. 1, pp. 29-35, January 2007.
[11] C. C. Liu and P. G. Schultz, “Adding New Chemistries to the Genetic Code,” Annu. Rev. Biochem., vol. 79, no. 1, pp. 413-444, June 2010.
[12] M. J. Schmidt and D. Summerer, “Genetic code expansion as a tool to study regulatory processes of transcription.,” Front. Chem., vol. 2, p. 7, 2014.
[13] W. H. Zhang, G. Otting, and C. J. Jackson, “Protein engineering with unnatural amino acids,” Curr. Opin. Struct. Biol., vol. 23, no. 4, pp. 581-587, August 2013.
[14] L. Wang, Z. Zhang, A. Brock, and P. G. Schultz, “Addition of the keto functional group to the genetic code of Escherichia coli,” Proc. Natl. Acad. Sci., vol. 100, no. 1, pp. 56-61, January 2003.
[15] H. Neumann, “Rewiring translation—Genetic code expansion and its applications,” FEBS Lett., vol. 586, no. 15, pp. 2057-2064, July 2012.
[16] K. Wals and H. Ovaa, “Unnatural amino acid incorporation in E. coli: current and future applications in the design of therapeutic proteins,” Front. Chem., vol. 2, p. 15, April 2014.
[17] T. Javahishvili et al., “Role of tRNA orthogonality in an expanded genetic code.,” ACS Chem. Biol., vol. 9, no. 4, pp. 874-9, April 2014.
[18] C. J. Noren, S. J. Anthony-Cahill, M. C. Griffith, and P. G. Schultz, “A general method for site-specific incorporation of unnatural amino acids into proteins.,” Science, vol. 244, no. 4901, pp. 182-8, April 1989.
[19] J. A. Johnson, Y. Y. Lu, J. A. Van Deventer, and D. A. Tirrell, “Residue-specific incorporation of non [symbol] canonical amino acids into proteins: recent developments and applications.”
[20] E. G. Worst et al., “Residue-specific Incorporation of Noncanonical Amino Acids into Model Proteins Using an Escherichia coli Cell-free Transcription-translation System Video Link,” J. Vis. Exp.
[21] A. Singh-Blom, R. A. Hughes, and A. D. Ellington, “Residue-Specific Incorporation of Unnatural Amino Acids into Proteins In Vitro and In Vivo,” in Methods in molecular biology (Clifton, N.J.), vol. 978,2013, pp. 93-114.
[22] S. H. Hong, Y.-C. Kwon, and M. C. Jewett, “Non-standard amino acid incorporation into proteins using Escherichia coli cell-free protein synthesis,” Front. Chem., vol. 2, p. 34, June 2014.
[23] J. Jaric and N. Budisa, “Design of Orthogonal Pairs for Protein Translation: Selection Systems for Genetically Encoding Noncanonical Amino Acids in E. coli,” Springer, Berlin, Heidelberg, 2015, pp. 71-82.
[24] W. Wan, J. M. Tharp, and W. R. Liu, “Pyrrolysyl-tRNA synthetase: An ordinary enzyme but an outstanding genetic code expansion tool,” Biochim. Biophys. Acta-Proteins Proteomics, vol. 1844, no. 6, pp. 1059-1070, June 2014.
[25] A. K. Antonczak et al., “Importance of single molecular determinants in the fidelity of expanded genetic codes,” Proc. Natl. Acad. Sci., vol. 108, no. 4, pp. 1320-1325, January 2011.
[26] T. Terada and S. Yokoyama, “Escherichia coli Cell-Free Protein Synthesis and Isotope Labeling of Mammalian Proteins,” in Methods in enzymology, vol. 565,2015, pp. 311-345.
[27] K. A. Calhoun and J. R. Swartz, “Energy Systems for ATP Regeneration in Cell-Free Protein Synthesis Reactions,” in In Vitro Transcription and Translation Protocols, vol. 375, Totowa, N.J.: Humana Press, 2007, pp. 3-17.
[28] J. W. Whittaker, “Cell-free protein synthesis: the state of the art.”
[29] D.-M. Kim and J. R. Swartz, “Regeneration of adenosine triphosphate from glycolytic intermediates for cell-free protein synthesis,” Biotechnol. Bioeng., vol. 74, no. 4, pp. 309-316, August 2001.
[30] K. A. Calhoun and J. R. Swartz, “Energizing cell-free protein synthesis with glucose metabolism,” Biotechnol. Bioeng., vol. 90, no. 5, pp. 606-613, June 2005.
[31] F. Caschera and V. Noireaux, “A cost-effective polyphosphate-based metabolism fuels an all E. coli cell-free expression system,” Metab. Eng., vol. 27, pp. 29-37, January 2015.
[32] A. S. M. Salehi, C. C. Earl, C. Muhlestein, and B. C. Bundy, “Escherichia coli-based cell-free extract development for protein-based cancer therapeutic production,” Int. J. Dev. Biol., vol. 60, no. 7-8-9, pp. 237-243, 2016.
[33] D.-M. Kim and J. R. Swartz, “Oxalate improves protein synthesis by enhancing ATP supply in a cell-free system derived from Escherichia coli,” Biotechnol. Lett., vol. 22, no. 19, pp. 1537-1542, 2000.
[34] M. C. Jewett, K. A. Calhoun, A. Voloshin, J. J. Wuu, and J. R. Swartz, “An integrated cell-free metabolic platform for protein production and synthetic biology,” Mol. Syst. Biol., vol. 4, p. 220, October 2008.
[35] M. C. Jewett, M. L. Miller, Y. Chen, and J. R. Swartz, “Continued protein synthesis at low [ATP] and [GTP] enables cell adaptation during energy limitation.,” J. Bacteriol., vol. 191, no. 3, pp. 1083-91, February 2009.
[36] M. C. Jewett and J. R. Swartz, “Mimicking the Escherichia coli cytoplasmic environment activates long-lived and efficient cell-free protein synthesis,” Biotechnol. Bioeng., vol. 86, no. 1, pp. 19-26, April 2004.
[37] L. Jiang, J. Zhao, J. Lian, and Z. Xu, “Cell-free protein synthesis enabled rapid prototyping for metabolic engineering and synthetic biology,” Synth. Syst. Biotechnol., vol. 3, no. 2, pp. 90-96, June 2018.
[38] X. Jiang, K. Oohira, Y. Iwasaki, H. Nakano, S. Ichihara, and T. Yamane, “Reduction of protein degradation by use of protease-deficient mutants in cell-free protein synthesis system of Escherichia coli.,” J. Biosci. Bioeng., vol. 93, no. 2, pp. 151-6, 2002.
[39] S. Leirmo, C. Harrison, D. S. Cayley, R. R. Burgess, and M. T. Record, “Replacement of potassium chloride by potassium glutamate dramatically enhances protein-DNA interactions in vitro,” Biochemistry, vol. 26, no. 8, pp. 2095-2101, April 1987.
[40] B. D. Bennett, E. H. Kimball, M. Gao, R. Osterhout, S. J. Van Dien, and J. D. Rabinowitz, “Absolute metabolite concentrations and implied enzyme active site occupancy in Escherichia coli,” Nat. Chem. Biol., vol. 5, no. 8, pp. 593-599, August 2009.
[41] T.-W. Kim, D.-M. Kim, and C.-Y. Choi, “Rapid production of milligram quantities of proteins in a batch cell-free protein synthesis system,” J. Biotechnol., vol. 124, no. 2, pp. 373-380, July 2006.
[42] A. V. Dobrynin and M. Rubinstein, “Theory of polyelectrolytes in solutions and at surfaces,” Prog. Polym. Sci., vol. 30, no. 11, pp. 1049-1118, November 2005.
[43] H. Morawetz, “Polyelectrolyte Solutions: Phenomena and Interpretation,” 2006, pp. 1-18.
[44] A. L. Hillberg and M. Tabrizian, “Biorecognition through Layer-by-Layer Polyelectrolyte Assembly: In-Situ Hybridization on Living Cells,” Biomacromolecules, vol. 7, no. 10, pp. 2742-2750, October 2006.
[45] J. Borges and J. F. Mano, “Molecular Interactions Driving the Layer-by-Layer Assembly of Multilayers,” Chem. Rev., vol. 114, no. 18, pp. 8883-8942, September 2014.
[46] K. Ariga, J. P. Hill, and Q. Ji, “Layer-by-layer assembly as a versatile bottom-up nanofabrication technique for exploratory research and realistic application,” Phys. Chem. Chem. Phys., vol. 9, no. 19, p. 2319, May 2007.
[47] L. L. del Mercato et al., “Biological applications of LbL multilayer capsules: From drug delivery to sensing,” Adv. Colloid Interface Sci., vol. 207, pp. 139-154, May 2014.
[48] J. Saqib and I. H. Aljundi, “Membrane fouling and modification using surface treatment and layer-by-layer assembly of polyelectrolytes: State-of-the-art review,” J. Water Process Eng., vol. 11, pp. 68-87, June 2016.
[49] S. Hajji et al., “Structural differences between chitin and chitosan extracted from three different marine sources,” Int. J. Biol. Macromol., vol. 65, pp. 298-306, April 2014.
[50] H. Ahlafi, H. Moussout, F. Boukhlifi, M. Echetna, M. N. Bennani, and S. My Slimane, “Kinetics of N-Deacetylation of Chitin Extracted from Shrimp Shells Collected from Coastal Area of Morocco,” Mediterr. J. Chem., vol. 2, no. 3, pp. 503-513, January 2013.
[51] S. Demirci, A. Alaslan, and T. Caykara, “Preparation, characterization and surface pKa values of poly(N-vinyl-2-pyrrolidone)/chitosan blend films,” Appl. Surf. Sci., vol. 255, no. 11, pp. 5979-5983, March 2009.
[52] T. Andersen, P. Auk-Emblem, and M. Dornish, “3D Cell Culture in Alginate Hydrogels,” Microarrays, vol. 4, no. 2, pp. 133-161, March 2015.
[53] J. P. Paques, E. Van Der Linden, C. J. M. Van Rijn, and L. M. C. Sagis, “Preparation methods of alginate nanoparticles,” Adv. Colloid Interface Sci., vol. 209, pp. 163-171, 2014.
[54] K. Y. Lee and D. J. Mooney, “Alginate: Properties and biomedical applications,” Prog. Polym. Sci., vol. 37, no. 1, pp. 106-126, January 2012.
[55] U. A. Shinde and M. S. Nagarsenker, “Characterization of gelatin-sodium alginate complex coacervation system.,” Indian J. Pharm. Sci., vol. 71, no. 3, pp. 313-7, May 2009.
[56] N. Malanovic, “Gram-positive bacterial cell envelopes: The impact on the activity of antimicrobial peptides,” Biochim. Biophys. Acta-Biomembr., vol. 1858, no. 5, pp. 936-946, May 2016.
[57] A. L. Hillberg and M. Tabrizian, “Biorecognition through Layer-by-Layer Polyelectrolyte Assembly: In-Situ Hybridization on Living Cells.”
[58] V. A. Fischetti, “Bacteriophage lysins as effective antibacterials.,” Curr. Opin. Microbiol., vol. 11, no. 5, pp. 393-400, October 2008.
[59] V. A. Fischetti, “Exploiting what phage have evolved to control gram-positive pathogens.,” Bacteriophage, vol. 1, no. 4, pp. 188-194, July 2011.
[60] I.-N. Wang, D. L. Smith, and R. Young, “Holins: The Protein Clocks of Bacteriophage Infections,” Annu. Rev. Microbiol., vol. 54, no. 1, pp. 799-825, October 2000.
[61] “Controlled lysis of bacteria,” August 2005.
[62] I. Young, I. Wang, and W. D. Roof, “Phages will out: strategies of host cell lysis.,” Trends Microbiol., vol. 8, no. 3, pp. 120-8, March 2000.
[63] Y. Shimizu, Y. Kuruma, T. Kanamori, and T. Ueda, “The PURE System for Protein Production,” in Methods in molecular biology (Clifton, N.J.), vol. 1118,2014, pp. 275-284.
[64] L. Achbergerová and J. Nahalka, “Polyphosphate—an ancient energy source and active metabolic regulator,” Microb. Cell Fact., vol. 10, no. 1, p. 63, August 2011.
[65] X. Zhang, H. Wu, B. Huang, Z. Li, and Q. Ye, “One-pot synthesis of glutathione by a two-enzyme cascade using a thermophilic ATP regeneration system,” J. Biotechnol., vol. 241, pp. 163-169, January 2017.
[66] Y. Chemla, E. Ozer, O. Schlesinger, V. Noireaux, and L. Alfonta, “Genetically expanded cell-free protein synthesis using endogenous pyrrolysyl orthogonal translation system,” Biotechnol. Bioeng., vol. 112, no. 8, pp. 1663-1672, August 2015.
[67] T. Yanagisawa, R. Ishii, R. Fukunaga, T. Kobayashi, K. Sakamoto, and S. Yokoyama, “Multistep Engineering of Pyrrolysyl-tRNA Synthetase to Genetically Encode Nε-(o-Azidobenzyloxycarbonyl) lysine for Site-Specific Protein Modification,” Chem. Biol., vol. 15, no. 11, pp. 1187-1197, November 2008.
[68] A. Ambrogelly, S. Gundllapalli, S. Herring, C. Polycarpo, C. Frauer, and D. Soll, “Pyrrolysine is not hardwired for cotranslational insertion at UAG codons,” Proc. Natl. Acad. Sci., vol. 104, no. 9, pp. 3141-3146, February 2007.
[69] T. R. Burke, M. S. Smyth, M. Nomizu, A. Otaka, and P. R. Roller, “Preparation of fluoro- and hydroxy-4-(phosphonomethyl)-D,L-phenylalanine suitably protected for solid-phase synthesis of peptides containing hydrolytically stable analogs of 0-phosphotyrosine,” J. Org. Chem., vol. 58, no. 6, pp. 1336-1340, March 1993.
[70] M. G. Macey, “Principles of Flow Cytometry,” in Flow Cytometry, Totowa, N.J.: Humana Press, pp. 1-15.
[71] V. A. Gant, G. Warnes “, I. Phillips, and G. F. Savidge, “The application of flow cytometry to the study of bacterial responses to antibiotics,” J. Med. Microbiol, vol. 39, pp. 147-154, 1993.
[72] K. J. Yong and D. J. Scott, “Rapid directed evolution of stabilized proteins with cellular high-throughput encapsulation solubilization and screening (CHESS),” Biotechnol. Bioeng., vol. 112, no. 3, pp. 438-446, March 2015.
[73] M. T. Smith, S. D. Berkheimer, C. J. Werner, and B. C. Bundy, “Lyophilized Escherichia coli-based cell-free systems for robust, high-density, long-term storage,” Biotechniques, vol. 56, no. 4, pp. 186-93, April 2014.
[74] D. Silva, J. Almeida, and R. de Oliveira, “The Use of Cationic Starch and Starch Complexes as Alternative to Improve Fiber Quality for Printing and Writing Grades.” 2011.
[75] M. H. Struszczyk “Chitin and chitosan—part I. Properties and production.” Polimery., vol. 47 pp. pp. 316-25, 2002.
[76] BolsterBio. (2018). FLOW CYTOMETRY PRINCIPLES. [online] Available at: https://www.facs-analysis.com/flow-cytometry-principle/[Accessed 26 May 2018].

1. A method of producing encapsulated cells, including the steps of:
(i) providing a plurality of microbial cells, the microbial cells modified to express a cytolytic enzyme;
(ii) contacting the microbial cells with a cationic polymer;
(iii) contacting the microbial cells with an anionic polymer;
to thereby produce encapsulated cells.

2. The method of claim 1, further including the step of facilitating at least partial hydrolysis of a cell wall of the microbial cells by the cytolytic enzyme.

3. The method of claim 2, wherein the step of facilitating hydrolysis of the cell wall comprises subjecting the encapsulated cells to a freeze-thaw cycle.

4. The method of claim 2, wherein the step of facilitating hydrolysis of the cell wall comprises lyophilisation of the encapsulated cells.

5. The method of claim 1, wherein the cationic polymer is or comprises chitosan.

6. The method of claim 1, wherein the anionic polymer is or comprises alginate.

7. The method of claim 1, wherein the microbial cells are or comprise E. coli.

8. The method of claim 1, wherein the cytolytic enzyme is or comprises an endolysin.

9. An encapsulated cell produced by the method of claim 1.

10. An encapsulated cell comprising a microbial cell modified to express a cytolytic enzyme, the microbial cell encapsulated with one or more layers of a cationic polymer and one or more layers of an anionic polymer.

11. The encapsulated cell of claim 10, wherein a cell al of the microbial cell has been at least partly hydrolysed by the cytolytic enzyme.

12. The encapsulated cell of claim 10, wherein the encapsulated cell has been subjected to a freeze-thaw cycle.

13. The encapsulated cell of claim 10, wherein the encapsulated cell is lyophilised.

14. The encapsulated cell of claim 10, wherein the cationic polymer is or comprises chitosan.

15. The encapsulated cell of claim 10, wherein the anionic polymer is or comprises alginate.

16. The encapsulated cell of claim 10, wherein the microbial cells are or comprise E. coli.

17. The encapsulated cell of claim 10, wherein the cytolytic enzyme is or comprises an endolysin.

18. (canceled)

19. (canceled)

20. (canceled)

21. (canceled)

22. (canceled)

23. (canceled)

24. The encapsulated cell of claim 11, wherein the encapsulated cell is further modified to comprise a nucleic acid sequence that encodes a target protein for synthesis.

25. The encapsulated cell of claim 11, wherein the encapsulated cell is further modified to comprise a nucleic acid sequence that encodes an enzyme that is configured to at least partly degrade a contaminant in a contaminated environment.

26. The encapsulated cell of claim 11, wherein the encapsulated cell is further modified to comprise a nucleic acid sequence that encodes a marker protein, wherein the nucleic acid sequence is configured to express the marker protein upon direct and/or indirect binding with an analyte in a sample.
